SEQUENCE LISTING (29 sequences)

SEQ ID NO: 1 - protein, 118 aa, Callithrix jacchus
QVQLVQSGAEVKKPGASVKVSCKASGYTFTSYAISWARQPPGQGLEWMGAFDPEYGSTTYAQKFQGRVTMTADTSTSTAYMELSSLRPEDTAVYYCARDVNFGNYFDYWGQGTLVTVS

SEQ ID NO: 2 - protein, 330 aa, Homo sapiens [sequence omitted]

SEQ ID NO: 3 - protein, 109 aa, Callithrix jacchus
ELVMTQSPATLSLSPGERATVSCRAGQSVSYYLAWYQQKPGQAPRLLIYGASTRATGIPARFSGSRSGTDFTLTISSLEPEDFAVYYCQQYSSWPPTFGQGTKLEIKRV

SEQ ID NO: 4 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 449 aa, Artificial Sequence
  AB138 heavy chain sequence
QVQLVQSGAEVKKPGASVKVSCKASGYTFTSYAISWARQPPGQGLEWMGAFDPEYGSTTYAQKFQGRVTMTADTSTSTAYMELSSLRPEDTAVYYCARDVNFGNYFDYWGQGTLVTVSSASTKNPDVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 214 aa, Artificial Sequence
  AB138 light chain sequence
ELVMTQSPATLSLSPGERATVSCRAGQSVSYYLAWYQQKPGQAPRLLIYGASTRATGIPARFSGSRSGTDFTLTISSLEPEDFAVYYCQQYSSWPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 117 aa, Mus musculus
QVQLQQSGAELVRPGTSVKVSCKASGYAFTNYLIEWVKQRPGQGLEWIGVINPGSGSTNYNEKFKDKATLTADKSSSTAYMQLSSLTSDDSAVYFCVRDYGYDGMDYWGQGTSVTVS

SEQ ID NO: 8 - protein, 114 aa, Mus musculus
DIVLTQSPASLAVSLGQRATISCRASKSVSTSGYSYMHWYQQKPGQPPKLLIYLASNLESGVPARFSGSGSGTDFTLNIHPVEEEDAATYYCQHSRELPLTFGAGTKLELKRTV

SEQ ID NO: 9 - protein, 118 aa, Homo sapiens
QVQLVQSGAEVKKPGASVKVSCKASGYTFTNYYMHWVRQAPGQGLEWMGIINPSGGSTDYAQNFQGRVTMTRDTSTSTVYMELTSLRSEDTAVYYCARIGVHDYWGQGTQVTVSSAST

SEQ ID NO: 10 - protein, 110 aa, Homo sapiens
EIVMTQSPATLSLSPGERATLSCRASQSVNNYLAWYQQRPGQAPSLLIYDASNRATGIPARFSGSGSGTDFTLTISSLEPEDFAVYYCQQRGNWPHTFGQGTKLEIKRTV

SEQ ID NO: 11 - protein, 117 aa, Artificial Sequence
  AB197 heavy chain sequence
QVQLVQSGAELKKPGASVKVSCKASGYAFTNYLIEWVRQPPGQGLEWIGVINPGSGSTNYNEKFKDKATLTADKSSSTAYMELSSLRPEDTAVYYCARDYGYDGMDYWGQGTLVTVS

SEQ ID NO: 12 - protein, 114 aa, Artificial Sequence
  AB197 light chain sequence
ELVMTQSPATLSLSPGERATVSCRASKSVSTSGYSYMHWYQQKPGQAPRLLIYLASNLESGVPARFSGSRSGTDFTLTISSLEPEDFAVYYCQHSRELPLTFGQGTKLEIKRTV

SEQ ID NO: 13 - protein, 10 aa, Artificial Sequence
  MOG specific IgG heavy chain CDR1 acceptor sequence
GYTFTSYAIS

SEQ ID NO: 14 - protein, 10 aa, Artificial Sequence
  AB164 heavy chain CRD1 donor sequence
GYAFTNYLIE

SEQ ID NO: 15 - protein, 17 aa, Artificial Sequence
  MOG specific IgG heavy chain CDR2 acceptor sequence
AFDPEYGSTTYAQKFQG

SEQ ID NO: 16 - protein, 17 aa, Artificial Sequence
  AB164 heavy chain CDR2 acceptor sequence
VINPGSGSTNYNEKFKD

SEQ ID NO: 17 - protein, 10 aa, Artificial Sequence
  MOG specific IgG heavy chain CDR3 acceptor sequence
DVNFGNYFDY

SEQ ID NO: 18 - protein, 9 aa, Artificial Sequence
  AB164 heavy chain CDR3 donor sequence
DYGYDGMDY

SEQ ID NO: 19 - protein, 11 aa, Artificial Sequence
  MOG specific IgG light chain CDR1 acceptor sequence
RAGQSVSYYLA

SEQ ID NO: 20 - protein, 15 aa, Artificial Sequence
  AB164 light chain CDR1 donor sequence
RASKSVSTSGYSYMH

SEQ ID NO: 21 - protein, 7 aa, Artificial Sequence
  MOG specific IgG light chain CDR2 acceptor sequence
GASTRAT

SEQ ID NO: 22 - protein, 7 aa, Artificial Sequence
  AB164 light chain CDR2 donor sequence
LASNLES

SEQ ID NO: 23 - protein, 9 aa, Artificial Sequence
  MOG specific IgG light chain CDR3 acceptor sequence
QQYSSWPPT

SEQ ID NO: 24 - protein, 9 aa, Artificial Sequence
  AB164 light chain CDR3 donor sequence
QHSRELPLT

SEQ ID NO: 25 - DNA, 6 bp, Artificial Sequence
  Kozak sequence
gccacc

SEQ ID NO: 26 - protein, 19 aa, Artificial Sequence
  human IgG gamma leader sequence
MEWSWVFLFFLSVTTGVHS

SEQ ID NO: 27 - protein, 20 aa, Artificial Sequence
  human kappa leader sequence
MSVPTQVLGLLLLWLTDARC

SEQ ID NO: 28 - protein, 75 aa, Artificial Sequence
  Consensus sequence
QVQLQSGAEPGSVKVSCKASGYFTYIWQPGQGLEWGPGSTYKFTTADSSTAYMLSSLDAVYCRDDYWGQGTVTVS

SEQ ID NO: 29 - protein, 71 aa, Artificial Sequence
  Consensus sequence
VTQSPALSGRATSCRASVSYWYQQKPGQPLLIYASGPARFSGSSGTDFTLIEEDAYYCQPTFGGTKLEKRV

(19) United States
(12) Patent Application Publication
(10) Pub. No.: US 2008/0095767 A1
(43) Pub. Date: Apr. 24, 2008

(54) ENGINEERED ANTIBODIES WITH NEW WORLD PRIMATE FRAMEWORK REGIONS

(75) Inventors: Philip A. JENNINGS, Warrawee (AU); Anthony G. Doyle, Drummoyne (AU); Adam W. Clarke, Russell Lea (AU); Robert D. Gay, North Bondi (AU)

Correspondence Address: MORRISON & FOERSTER LLP, 755 PAGE MILL RD, PALO ALTO, CA, 94304-1018, US

(73) Assignee: Peptech Limited, Macquarie Park (AU)

(21) Appl. No.: 11/832,553

(22) Filed: Aug. 1, 2007

Related U.S. Application Data
(63) Continuation of application No. PCT/AU06/01165, filed on Aug. 15, 2006.
(60) Provisional application No. 60/709,333, filed on Aug. 17, 2005.

(30) Foreign Application Priority Data
Aug. 15, 2005 (AU) 2005904406

(51) Int. Cl. A61K 39/395; C07K 16/18

(52) U.S. Cl. 424/133.1; 530/387.3

(57) ABSTRACT

The present invention provides an antibody or antigen-binding portion thereof having a variable region comprising at least two complementarity determining regions (CDRs) and at least three framework regions. The the framework regions are, or are derived from New World primate framework regions, and at least one of the CDRs is a non-New World primate CDR.

### FIELD OF THE INVENTION

The present invention relates to an antibody or antigen-binding portion thereof having a variable region comprising at least two complementarity determining regions (CDRs) and at least three framework regions. The framework regions are, or are derived from New World primate framework regions, and at least one of the CDRs is either a modified New World primate CDR or a non-New World primate CDR.

### BACKGROUND OF THE INVENTION

Antibodies (immunoglobulins) play an important role in the immune system of a mammal. They are produced by plasma cells which have developed from precursor B cells. Antibodies consist of two identical light polypeptide chains and two identical heavy polypeptide chains which are joined by disulfide bridges. The light chains are referred to as either kappa or lambda light chains and the heavy chains as gamma, mu, delta, alpha or epsilon. Each chain consists of a constant and variable region. The variable region gives the antibody is specificity. Within each variable region are regions of hypervariability or complementarity determining regions (CDRs) which are flanked by more conserved regions referred to as framework regions. Within each variable region are three CDRs and four framework regions.

Antibodies are bifunctional molecules, the N-terminal variable segments from the heavy and light chains associate together in a specific manner to generate a three-dimensional structure with affinity for a particular epitope of the surface of an antigen. The constant region segments are responsible for prolonged serum half-life and the effector functions of the antibody and relate to complement binding, stimulation of phagocytosis, antibody-dependent cellular cytotoxicity and triggering of granulocyte granule release.

The development of hybridoma technology has facilitated the production of monoclonal antibodies of a particular specificity. Typically, such hybridomas are murine hybridomas.

Human/mouse chimeric antibodies have been created in which antibody variable region sequences from the mouse genome are combined with antibody constant region sequences from the human genome. The chimeric antibodies exhibit the binding characteristics of the parental mouse antibody, and the effector functions associated with the human constant region. The antibodies are produced by expression in a host cell, including for example Chinese Hamster Ovary (CHO), NS0 myeloma cells, COS cells and SP2 cells.

Such chimeric antibodies have been used in human therapy, however antibodies to these chimeric antibodies have been produced by the human recipient. Such anti-chimeric antibodies are detrimental to continued therapy with chimeric antibodies.

It has been suggested that human monoclonal antibodies are expected to be an improvement over mouse monoclonal antibodies for in vivo human therapy. From work done with antibodies from Old World primates (rhesus monkeys and chimpanzees) it has been postulated that these non-human primate antibodies will be tolerated in humans because they are structurally similar to human antibodies (Ehrlich P H et al., Clin Chem., 1988, 34:9 1681-1688). Furthermore, because human antibodies are non-immunogenic in Rhesus monkeys (Ehrich P H et al., Hybridoma, 1987, 6:151-60), it is likely that the converse is also applicable and primate antibodies will be non-immunogenic in humans. These monoclonal antibodies are secreted by hybridomas constructed by fusing lymphocytes to a human x mouse heteromyeloma.

EP 0 605 442 disclosed chimeric antibodies which bind human antigens. These antibodies comprise the whole variable region from an Old World monkey and the constant region of a human or chimpanzee antibody. One of the advantages suggested in this reference for these constructs is the ability to raise antibodies in Old World monkeys to human antigens which are less immunogenic in humans compared with antibodies raised in a mouse host.

New World primates (infraorder—Platyrrhini) comprises at least 53 species commonly divided into two families, the Callithricidae and Cebidae. The Callithricidae consist of marmosets and tamarins. The Cebidae includes the squirrel monkey, titi monkey, spider monkey, woolly monkey, capuchin, uakaris, sakis, night or owl monkey and the howler monkey.

Evolutionarily distant primates, such as New World primates, are not only sufficiently different from humans to allow antibodies against human antigens to be generated, but are sufficiently similar to humans to have antibodies similar to human antibodies so that the host does not generate an anti-antibody immune response when such primate-derived antibodies are introduced into a human.

Previous studies have characterised the expressed immunoglobulin heavy chain repertoire of the Callithrix jacchus marmoset (von Budingen H-C et al., Immunogenetics 2001, 53:557-563). Six IGHV subgroups were identified which showed a high degree of sequence similarity to their human IGHV counterparts. The framework regions were more conserved when compared to the complementarity determining regions (CDRs). The degree of similarity between C. jacchus and human IGHV sequences was less than between non-human Old World primates and humans.

Domain Antibodies

Domain antibodies (dAb) are functional binding units which can be created using antibody frameworks and correspond to the variable regions of either the heavy (V_{H}) or light (V_{L}) chains of antibodies. Domain antibodies have a molecular weight of approximately 13 kDa, or less than one tenth the size of a full antibody.

Immunoglobulin light chains are referred to as either kappa or lambda light chains and the heavy chains as gamma, mu, delta, alpha or epsilon. The variable region gives the antibody its specificity. Within each variable region are regions of hypervariability, otherwise known as complementarity determining regions (CDRs) which are flanked by more conserved regions referred to as framework regions. Within each light and heavy chain variable region are three CDRs and four framework regions.

In contrast to conventional antibodies, domain antibodies are well expressed in bacterial, yeast and mammalian systems. Their small size allows for higher molar quantities per gram of product, thus providing a significant increase in potency. In addition, domain antibodies can be used as a building block to create therapeutic products such as multiple targeting dAbs in which a construct containing two or more variable domains bind to two or more therapeutic targets, or dAbs targeted for pulmonary or oral administration.

### SUMMARY OF THE INVENTION

The present inventors have found that New World primates provide a source of antibody sequences which are predicted to have low immunogenicity in humans.

New world primates were chosen as a repository of immunoglobulin sequences that existed at the branch point of New World and Old World Primates. The key idea was that this repository might thus yield immunoglobulin sequences primordial to later divergences in immunoglobulin sequences as found in Old World Primates. Such primordial sequences would have co-existed with the T cell repertoire, as it subsequently evolved on the path to man, for the 35 million years ago (MYA) estimated to be the branch point of Old and New World Primates (Schneider H et al, Mol Phylogenet Evol., 1993 Sep.; 2(3):225-42). This represents a protracted period of selection for immunological tolerance and thus such primordial sequences were predicted, by the inventors, to be free of certain helper T cell epitopes that would have evolved more recently.

Accordingly in a first aspect the present invention provides an antibody or antigen-binding portion thereof having a variable region comprising at least two complementarity determining regions (CDRs) and at least three framework regions, wherein the framework regions are, or are derived from New World primate framework regions, and wherein at least one of the CDRs is a non-New World primate CDR.

In a second aspect, the invention provides a pharmaceutical composition comprising an effective amount of the antibody or antigen-binding portion thereof according to the present invention, together with a one or more pharmaceutically acceptable excipient(s) or diluent(s).

In a third aspect, the invention provides for the use of an antibody or antigen-binding portion thereof of the present invention in a diagnostic application for detecting an antigen associated with a particular disease or disorder.

In a fourth aspect, the present invention provides a method for treating a disease or disorder characterised by human TNF-α activity in a human subject, comprising administering to the subject in need thereof an effective amount of the antibody or antigen binding portion thereof as described herein (or a pharmaceutical composition thereof) in which the antibody or antigen-binding portion thereof binds TNF-α.

In a further aspect of the invention is provided the use of the antibodies, and antigen binding portions thereof, and pharmaceutical compositions thereof as described herein in the manufacture of a medicament. Particularly, the manufacture of a medicament for use in the treatment or diagnosis of diseases or disorders as described herein.

In a further aspect the present invention provides a designed New World primate antibody or antigen-binding portion thereof which binds a cell surface antigen or a cytokine wherein the antibody or antigen-binding thereof comprises a variable region comprising at least two complementarity determining regions (CDRs) and at least three framework regions, wherein the CDRs are selected such that the antibody or antigen-binding portion binds to the cell surface antigen or to the cytokine.

Unless otherwise noted or clearly indicated in by the context, it is intended that the antibodies and antigen binding portions thereof as described herein may be used without limitation in the pharmaceutical compositions described herein and incorporated in the kits described herein. And, further the antibodies and antigen binding portions thereof, as well as the pharmaceutical compositions and kits, as described herein may be used in the methods of treatment and diagnosis disclosed herein, unless otherwise noted or clearly indicated by the context.

### BRIEF DESCRIPTION OF THE FIGURES

FIG. 1 demonstrates the binding of AB138 to rat MOG present in rat spinal cord lysate (lane 2) and not to CHOK1SV lysate (lane 3). Lane 1 contains molecular weight markers.

FIG. 2 demonstrates the lack of non-specific binding of an anti-TNFα monoclonal antibody to the same sample of rate MOG present in rat spinal cord lysate (lane 2) and CHOK1SV lysate (lane 3). Lane 1 contains molecular weight markers.

FIG. 3 is an alignment of the donor and acceptor V_{H} amino acid sequences

FIG. 4 is an alignment of the donor and acceptor V_{L} amino acid sequences

FIG. 5: Binding of antibodies AB164, AB103 and AB197 to TNF-α by ELISA.

FIG. 6: Neutralisation by AB164, AB197, AB103 of TNF-α-induced L-929 cell cytotoxicity

### DETAILED DESCRIPTION OF THE INVENTION

In a first aspect the present invention provides an antibody or antigen-binding portion thereof having a variable region comprising at least two complementarity determining regions (CDRs) and at least three framework regions, wherein the framework regions are, or are derived from New World primate framework regions, and wherein at least one of the CDRs is a non-New World primate CDR.

In a second aspect, the invention provides a pharmaceutical composition comprising an effective amount of the antibody or antigen-binding portion thereof according to the present invention, together with a one or more pharmaceutically acceptable excipient(s) or diluent(s).

In a third aspect, the invention provides for the use of an antibody or antigen-binding portion thereof of the present invention in a diagnostic application for detecting an antigen associated with a particular disease or disorder.

In a fourth aspect, the present invention provides a method for treating a disease or disorder characterised by human TNF-α activity in a human subject, comprising administering to the subject in need thereof an effective amount of the antibody or antigen binding portion thereof as described herein (or a pharmaceutical composition thereof) in which the antibody or antigen-binding portion thereof binds TNF-α.

In certain embodiments of the invention the variable region comprises three CDRs and four framework regions. it is also preferred that the antibody has low predicted immunogenicity in humans.

The variable region of the antibody or antigen-binding portion thereof may comprise a combination of CDRs from differing sources.

In certain embodiments the variable region comprises CDRs selected from the group consisting of at least one murine CDR sequence (preferably either mouse or rat), at least one human CDR sequence, at least one synthetic CDR sequence, at least one rabbit CDR sequence, at least one modified New World primate CDR sequence and combinations of two or more of the forgoing, at least one human CDR and at least one murine CDR, at least one human CDR and at least one synthetic CDR, at least one human CDR and at least one rabbit CDR, at least one human CDR and at least one New World primate CDR, at least one murine CDR and at least one synthetic CDR, at least one murine CDR and at least one rabbit CDR, at least one murine CDR and at least one New World primate CDR, at least one synthetic CDR and at least one rabbit CDR, at least one synthetic CDR and at least one New World primate CDR, and at least one rabbit CDR and at least one New World primate CDR.

In a preferred form the variable region comprises 3 murine CDR sequences, in particular 3 mouse CDR sequences.

In an alternative embodiment the variable region comprises 3 human CDR sequences.

In a further preferred embodiment the variable region comprises 4 New World primate framework regions or 4 framework regions in which the regions are derived from New World primate framework regions.

In some embodiments the antigen-binding portion is a domain antibody. In particular embodiments, the antibody or antigen-binding portion further comprises a human or non-human Old World primate constant region sequence or a combination thereof.

Examples of non-human Old World primates include, but are not limited to, chimpanzees, baboons, orang utans, macaques and gorillas.

In a further embodiment of the present invention, the dAb may be multimerised, as for example, hetero- or homodimers (e.g., V_{H}/V_{H}, V_{L}/V_{L} or V_{H}/V_{L}), hetero- or homotrimers (e.g., V_{H}/V_{H}/V_{H}, V_{L}/V_{L}/V_{L}, V_{H}/V_{H}/V_{L} or V_{H}/V_{L}/V_{L}), hetero- or homotetramers (e.g., V_{H}/V_{H}/V_{H}/V_{H}, V_{L}/V_{L}/V_{L}/V_{L}, V_{H}/V_{H}/V_{H}/V_{L}, V_{H}/V_{H}/V_{L}/V_{L} or V_{H}/V_{L}/V_{L}/V_{L}), or higher order hetero- or homomultimers. Multimerisation can increase the strength of antigen binding, wherein the strength of binding is related to the sum of the binding affinities of the multiple binding sites.

For example, the invention provides a domain antibody wherein the domain antibody is linked to at least one further domain antibody. Each dAb may bind to the same or different antigens.

The dAb multimers may further comprise one or more dAbs which are linked and wherein each dAb binds to a different antigen multi-specific ligands including so-called “dual-specific ligands”. For example, the dual specific ligands may comprise a pair of V_{H} domains or a pair of V_{L} domains. Such dual-specific ligands are described in WO 2004/003019 (PCT/GB2003/002804) in the name of Domantis Ltd, incorporated by reference herein in its entirety.

The New World primate framework region sequence is preferably from a New World primate selected from the group consisting of marmosets, tamarins, squirrel monkey, titi monkey, spider monkey, woolly monkey, capuchin, uakaris, sakis, night or owl monkey and the howler monkey, most preferably a marmoset.

Preferably, the antigen to which the chimeric antibody or antigen-binding portion thereof binds, is peptide, protein, carbohydrate, glycoprotein, lipid or glycolipid in nature, selected from a tumour-associated antigen including carcinoembryonic antigen, EpCAM, Lewis-Y, Lewis-Y/b, PMSA, CD20, CD30, CD33, CD38, CD52, CD154, EGF-R, Her-2, TRAIL and VEGF receptors, an antigen involved in an immune or inflammatory disease or disorder including CD3, CD4, CD25, CD40, CD49d, MHC class I, MHC class II, GM-CSF, interferon-γ, IL-1, IL-12, IL-13, IL-23, TNF-α, and IgE, an antigen expressed on a host cell including glycoprotein IIb/IIIa, P-glycoprotein, purinergic receptors and adhesion receptors including CD11a, CD11b, CD11c, CD18, CD56, CD58, CD62 or CD144, an antigen comprising a cytokine, chemokine, growth factor or other soluble physiological modulator or a receptor thereof including eotaxin, IL-6, IL-8, TGF-β, C3a, C5a, VEGF, NGF and their receptors, an antigen involved in central nervous system diseases or disorders including β-amyloid and prions, an antigen of non-human origin such as microbial, nanobial or viral antigens or toxins including respiratory syncitial virus protein F, anthrax toxin, rattle snake venom and digoxin; wherein the chimeric antibody acts as an agonist or antagonist or is active to either deplete (kill or eliminate) undesired cells (eg. anti-CD4) by acting with complement, or killer cells (eg. NK cells) or is active as a cytotoxic agent or to cause Fc-receptor binding by a phagocyte or neutralizes biological activity of its target.

It is also preferred that the sequence of at least one framework region is modified to increase binding or potency or to decrease predicted immunogenicity in humans. An increase in binding or potency or a decrease in predicted immunogenicity in humans of an antibody or antigen-binding portion of the invention is relative to an antibody or antigen binding portion in which the framework region is unmodified.

In other embodiments the sequence of one or more of the CDRs are modified to increase binding or potency or to decrease predicted immunogenicity in humans. An increase in binding or potency or a decrease in predicted immunogenicity in humans of an antibody or antigen-binding portion of the invention is relative to an antibody or antigen binding portion in which the framework region is unmodified.

An increase in binding is demonstrated by a decrease in K_{D} (K_{off}/Kₒₙ) for the antibody or antigen binding portion thereof. An increase in potency is demonstrated in biological assays. For example, assays that can be used to measure the potency of the antibody or antigen-binding portion thereof include the TNFα-induced L929 cytotoxicity neutralisation assay, IL-12-induced human PHA-activated peripheral blood mononuclear cell (PBMC) proliferation assay, and RANKL mediated osteoclast differentiation of mouse splenocytes (Stern, Proc. Natl. Acad. Sci. USA 87:6808-6812 (1990); Kong, Y-Y. et al. Nature 397:315-323 (1990); Matthews, N. and M. L. Neale in Lymphokines and Interferons, a Practical Approach, 1987, M. J. Clemens, A. G. Morris and A. J. H. Gearing, eds., IRL Press, p. 221)

The term “antibody” as used herein, is intended to refer to immunoglobulin molecules comprised of four polypeptide chains, two heavy (H) chains and two light (L) chains interconnected by disulfide bonds. Each heavy chain is comprised of a heavy chain variable region (HCVR or V_{H}) and a heavy chain constant region. The heavy chain constant region comprises three domains, C_{H}1, C_{H}2 and C_{H}3. Each light chain is comprised of a light chain variable region (LCVR or V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4.

The term “antigen-binding portion” of an antibody, as used herein refers to one or more components or derivatives of an immunoglobulin that exhibit the ability to bind to an antigen. It ahs been shown that the antigen-binding function of an antibody can be performed by fragments of a full length antibody. Examples of binding fragments encompassed within the term “antigen-binding portion” of an antibody include (i) a Fab fragment, a monovalent fragment consisting of the V_{L}, V_{H}, C_{L} and C_{H}1 domains; (ii) a F(ab′)₂ fragment, a bivalent fragment comprising two Fab fragments linked by a disulfide bridge att he hinge region; (iii) a Fd fragment consisting of the V_{H} and C_{H}1 domains; (iv) a Fv fragment consisting of the V_{L} and V_{H} domains of a single arm of an antibody; (v) a dAb fragment (Ward et al, 1989, Nature 341:544-546) which consists of a single V_{H} domain, or a V_{L} domain (van den Beuken T et al, 2001, J. Mol. Biol. 310, 591); and (vi) an isolated complementarity determining region (CDR). Furthermore, although the two domains of the Fv fragment V_{L} and V_{H}, are coded by separate genes, they can be joined, using recombinant methods, by a synthetic linker that enables them to be made as a single protein chain in which the V_{L} and V_{H} regions pair to form monovalent molecules (known as single chain Fv (scFv); (see eg Bird et al., 1988, Science 242:423-426 and Huston et al., 1988 Proc. Natl. Acad. Sci. USA 85:5879-5883). Such single chain Fvs are also intended to be encompassed within the term “antigen-binding portion” of an antibody. Other forms of single chain Fvs and related molecules such as diabodies or triabodies are also encompassed. Diabodies are bivalent antibodies in which V_{H} and V_{L} domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al., 1993, Proc. Natl. Acad. Sci. USA, 90:6444-6448; Poljak, R. J., et al., 1994, Structure, 2:1121-1123).

Methods of producing antibodies according to the invention will be familiar to persons skilled in the art, see for example, U.S. Pat. No. 4,816,567, U.S. Pat. No. 5,585,089 and US 20030039649 which are incorporated herein by reference in their entirety. Such methods require the use of standard recombinant techniques.

It is preferred that the antibody or antigen-binding portion thereof according to the present invention has predicted low immunogenicity in a human host.

By “low immunogenicity” it is meant that the antibody does not raise an antibody response in at least the majority of individuals receiving the antibody of sufficient magnitude to reduce the effectiveness of continued administration of the antibody for a sufficient time to achieve therapeutic efficacy.

The level of immunogenicity in humans may predicted using the MHC class II binding prediction program Propred (http://www.imtech.res.in/raghava/propred) using a 1% threshold value analysis of all alleles. Other programs which may be used include:

Rankpep (http://bio.dfci.harvard.edu/Tools/rankpep.html)

Epibase (Algonomics proprietary software: algonomics.com)

Reduced immunogenicity molecules will contain no or a reduced numbers of peptides predicted to bind to MHC class II alleles that are highly expressed in the target population, relative to the starting donor molecule (Flower D R, Doytchinova I. A. (2004) Immunoinformatics and the prediction of immunogenicity, Drug Discov Today, 9(2): 82-90).

Functional analysis of MHC class II binding can be performed by generating overlapping peptides corresponding to the protein of interest and testing these for their ability to evoke T cell activation (T cell proliferation assay) or displace a reporter peptide, a known MHC class II-binding peptide (Hammer J et al., 1994, J. Exp. Med., 180:2353).

The term “derived from” as used herein in relation to New World primate framework regions means that the sequence of the New World primatic framework region is altered from the native sequence. Typically the changes will be made to increase binding such as described in U.S. Pat. No. 5,585,089 and US 20030039649 or to reduce predicted immunogenicity in humans: The term “derived from” does not include changes which result in the total sequence of the framework regions present in the variable region being identical to a human framework sequences. One database which may be used for comparison is http://www.ncbi.nlm.nih.gov/.

In a further aspect the present invention provides a designed New World primate antibody or antigen-binding portion thereof which binds a cell surface antigen or a cytokine wherein the antibody or antigen-binding thereof comprises a variable region comprising at least two complementarity determining regions (CDRs) and at least three framework regions, wherein the CDRs are selected such that the antibody or antigen-binding portion binds to the cell surface antigen or to the cytokine.

As used herein the term “designed” means the New World primate CDRs have been selected using the epitope imprinting methods described in Hoogenboom et al., PCT Publication No. WO 93/06213 and Jespers et al, BIO/TECHNOLOGY Vol 12 1994, pp 899-903 which are hereby incorporated in their entirety. The antibody libraries used in this method are preferably scFv libraries prepared and screened as described in McCafferty et al., PCT Publication No. WO 92/01047, McCafferty et al., 1990, Nature, 348:552-554; and Griffiths et al., 1993, EMBO J, 12:725-734 which are hereby incorporated by reference in their entirety.

For example, once initial human V_{L and V}_{H} segments are selected, “mix and match” experiments, in which different pairs of the initially selected V_{L} and V_{H} segments are screened for hTNF-α binding, are performed to select preferred V_{L}/V_{H} pair combinations. Additionally, to further improve the affinity and/or lower the off rate constant for hTNF-α binding, the V_{L} and V_{H} segments of the preferred V_{L}/V_{H} pair(s) can be randomly mutated, preferably within the CDR3 region of V_{H} and/or V_{L}, in a process analogous to the in vivo somatic mutation process responsible for affinity maturation of antibodies during a natural immune response. This in vitro affinity maturation can be accomplished by amplifying V_{H} and V_{L} regions using PCR primers complimentary to the V_{H} CDR3 or V_{L} CDR3, respectively, which primers have been “spiked” with a random mixture of the four nucleotide bases at certain positions such that the resultant PCR products encode V_{H} and V_{L} segments into which random mutations have been introduced into the V_{H} and/or V_{L} CDR3 regions. These randomly mutated V_{H} and V_{L} segments can be rescreened for binding to the antigen and sequences that exhibit high affinity and a low off rate for antigen binding can be selected.

Following screening and isolation of an antibody or antigen-binding portion thereof which binds the antigen of interest from a recombinant immunoglobulin display library, nucleic acid encoding the selected antibody can be recovered from the display package (e.g., from the phage genome) and subcloned into other expression vectors by standard recombinant DNA techniques. If desired, the nucleic acid can be further manipulated to create other antibody forms of the invention (e.g., linked to nucleic acid encoding additional immunoglobulin domains, such as additional constant regions). To express a recombinant human antibody isolated by screening of a combinatorial library, the DNA encoding the antibody is cloned into a recombinant expression vector and introduced into a mammalian host cells.

Examples of cell surface antigens which may be targeted and antibodies which may be used in the imprinting include but are not limited to

| Antigen | Antibody (reference) |
|---|---|
| CD3 | OKT3 (Van Wauwe-J P et al (1980) Journal |
| | of Immunology 124: 2708-13) |
| CD20 | 1F5 (Press-O W et al (1987) Blood 69: 584 |
| | Y2B8 (White-C A et al (1991) Pharm. Sci. |
| | Technol. Today 2: 95-101 |
| CD33 | P67.6 (Koller-U & Peschel-CH. In Knapp- |
| | W et al Eds Leukocyte Typin IV: White |
| | Cell Differentiation Antigens, Oxford |
| | University Press 1989: 812-813 |
| CD52 | CAMPATH 1 (Hale-G et al (1983) Blood |
| | 62: 873-82) |
| EGF-R | mAb225 (Bruell-D et al (2005) Int J Mol |
| | Med 15: 303-313) |
| Glycoprotein IIb/IIIa | 10E5 & 7E3 (Coller-B S (1985) Journal of |
| | Clinical Investigation 76: 101-108) |
| Her-2 | 4D5 (Kumar-R et al (1991) Mol. Cell Biol |
| | 11: 979-86) |
| CD25 | Mab: RFT5 (Engert-A et al (1991) Int J |
| | Cancer 49: 450-456) |

Examples of cytokines which may be targeted and antibodies which may be used in the imprinting include but are not limited to

| Antigen | Antibody (reference) |
|---|---|
| TNF-α | mAb195 (Moller-A et al (1990) Cytokine |
| | 2: 162-169) |
| | mAb1, 11, 12, 20, 21, 25, 31, 32, 37, 42, 47, |
| | 53, 54 (Rathjen D A et al (1991) Molecular |
| | Immunology 28: 79-86) |
| VEGF | mAbs A3.13.1, A4.6.1, B4.3.1, & B2.6.2 |
| | (Kim-K J (1992) Growth Factors 7: 53-64) |

The present invention if further based on a method for amplification of New World primate immunoglobulin genes, for example by polymerase chain reaction (PCR) from nucleic acid extracted from New World primate lymphocytes using primers specific for heavy and light chain variable region gene families. The amplified variable region is then cloned into an expression vector containing a human or primate constant region gene for the production of New World primate chimeric recombinant antibody. Standard recombinant DNA methodologies are used to obtain antibody heavy and light chain genes, incorporate these genes into recombinant expression vectors and introduce the vectors into host cells, such as those described in Sambrook, Fritsch and Maniatis (eds), Molecular Cloning: a laboratory manual, second edition, Cold Spring Harbor, N.Y. (1989).

Suitable expression vectors will be familiar to those skilled in the art. The New World primate lymphocytes producing the immunoglobulins are typically immortalised by fusion with a myeloma cell line to generate a hybridoma.

Preferred mammalian host cells for expressing the recombinant antibodies of the invention include Chinese Hamster Ovary (CHO), NS0 myeloma cells, COS cells and SP2 cells.

In addition to mammalian expression systems, the present invention also contemplates the use of non-mammalian expression systems such as those which are plant or prokaryotic (bacterial) derived. Such expression systems would be familiar to persons skilled in the art.

The repertoire of V_{H}, V_{L} and constant region domains can be a naturally occurring repertoire of immunoglobulin sequences or a synthetic repertoire. A naturally occurring repertoire is one prepared, for example, from immunoglobulin expressing cells harvested from one or more primates. Such repertoires can be naïve ie. prepared from newborn immunoglobulin expressing cells, or rearranged ie. prepared from, for example, adult primate B cells. If desired, clones identified from a natural repertoire, or any repertoire that bind the target antigen are then subject to mutagenesis and further screening in order to produce and select variants with improved binding characteristics.

Synthetic repertoires of immunoglobulin variable domains are prepared by artificially introducing diversity into a cloned variable domain. Such affinity maturation techniques will be familiar to persons skilled in the art such as those described by R. A. Irving et al., 2001, Journal of Immunological Methods, 248, 31-45.

The variable region, or a CDR thereof, of a New World primate antibody gene may be cloned by providing nucleic acid eg. cDNA, providing a primer complementary to the cDNA sequence encoding a 5′ leader sequence of an antibody gene, contacting the cDNA and the primer to form a hybrid complex and amplifying the cDNA to produce nucleic acid encoding the variable region (or CDR region) of the New World primate antibody gene.

In view of the teaching of the present specification, it will be appreciated by persons skilled in the art of the present invention, that New World primate variable region sequence may be used as acceptors for the grafting of non-New World primate sequences, in particular, CDR sequences using standard recombinant techniques. For example, U.S. Pat. No. 5,585,089 describes methods for creating low immunogenicity chimeric antibodies that retain the high affinity of the non-human parent antibody and contain one or more CDRs from a donor immunoglobulin and a framework region from a human immunoglobulin. United States publication no. 20030039649 describes a humanisation method for creating low immunogenicity chimeric antibodies containing CDR sequences from a non-human antibody and framework sequences of human antibodies based on using canonical CDR structure types of the non-human antibody in comparison to germline canonical CDR structure types of human antibodies as the basis for selecting the appropriate human framework sequences for a humanised antibody. Accordingly, these principles can be applied to the grafting of one or more non-New World primate CDRs into a New World primate acceptor variable region.

The CDR sequences may be obtained from the genomic DNA isolated from an antibody, or from sequences present in a database e.g. The National Centre for Biotechnology Information protein and nucleotide databases, The Kabat Database of Sequences of Proteins of Immunological Interest. The CDR sequence may be a genomic DNA or a cDNA.

Methods for grafting a replacement CDR(s) into an acceptor variable sequence will be familiar to persons skilled in the art of the present invention. Typically, the CDRs will be grafted into acceptor variable region sequences for each of a variable light chain and a variable heavy chain or a single chain in the case of a domain antibody. The preferred method of the present invention involves replacement of either CDR1 or, more preferably, CDR2 in a variable region sequence via primer directed mutagenesis. The method consists of annealing a synthetic oligonucleotide encoding a desired mutation to a target region where it serves as a primer for initiation of DNA synthesis in vitro, extending the oligonucleotide by a DNA polymerase to generate a double-stranded DNA that carries the desired mutation, and ligating and cloning the sequence into an appropriate expression vector (Sambrook, Joseph; and David W. Russell (2001), Molecular Cloning: A Laboratory Manual, 3rd ed., Cold Spring Harbor, N.Y.: Cold Spring Harbor Laboratory Press).

Still further, an antibody or antigen-binding portion thereof may be part of a larger immunoadhesion molecule, formed by covalent or noncovalent association of the antibody or antibody portion with one or more other proteins or peptides. Examples of such immunoadhesion molecules include use of the streptavidin core region to make a tetrameric scFv molecule (Kipriyanov, S. M., et al., 1995 Human Antibodies and Hybridomas, 6:93-101) and use of a cysteine residue, a marker peptide and a C-terminal polyhistidine tag to make bivalent and biotinylated scFv molecules (Kipriyanov, S. M., et al., 1994 Mol. Immunol., 31:1047-1058). Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques, as described herein and known to the skilled artisan.

The constant region sequence (Fc portion) is preferably obtained from a human or primate immunoglobulin sequence. The primate sequence may be a New World primate or an Old World primate sequence. Suitable Old World primates include chimpanzee, or other hominid ape eg. gorilla or orang utan, which because of their close phylogenetic proximity to humans, share a high degree of homology with the human constant region sequence. Sequences which encode for human or primate constant regions are available from databases including e.g. The National Centre for Biotechnology Information protein and nucleotide databases, The Kabat Database of Sequences of Proteins of Immunological Interest.

The antibody or antigen-binding portion according tot he invention is capable to binding to a human or non-human antigen.

Preferably, the antigen to which the chimeric antibody or antigen-binding portion thereof binds, is peptide, protein, carbohydrate, glycoprotein, lipid or glycolipid in nature, selected from a tumour-associated antigen including carcinoembryonic antigen, EpCAM, lewis-Y, Lewis-Y/b, PMSA, Cd20, CD30, CD33, CD38, CD52, CD154, EGF-R, Her-2, TRAIL and VEGF receptors, an antigen involved in an immune or inflammatory disease or disorder including CD3, CD4, CD25, CD40, CD49d, MHC class I, MHC class II, GM-CSF, interferon-γ, IL-1, IL-12, IL-13, IL-23, TNF-α, and IgE, an antigen expressed on a host cell including glycoprotein IIb/IIIa, P-glycoprotein, purinergic receptors and adhesion receptors including CD11a, CD11b, CD11c, CD18, CD56, CD58, CD62 or CD144, an antigen comprising a cytokine, chemokine, growth factor or other soluble physiological modulator or a receptor thereof including eotaxin, IL-6, IL-8, TGF-β, C3a, C5a, VEGF, NGF and their receptors, an antigen involved in central nervous system diseases or disorders including β-amyloid and prions, an antigen of non-human origin such as microbial, nanobial or viral antigens or toxins including respiratory syncitial virus protein F, anthrax toxin, rattle snake venom and digoxin; wherein the chimeric antibody acts as an agonist or antagonist or is active to either deplete (kill or eliminate) undesired cells (eg. anti-CD4) by acting with complement, or killer cells (eg. NK cells) or is active as a cytotoxic agent or to cause Fc-receptor binding by a phagocyte or neutralizes biological activity of its target.

More preferably, the antigen is TNFα, most preferably human TNFα.

Alternatively the antibody or antigen-binding portion thereof may bind a non-human antigen. Preferably the non-human antigen is selected from the group consisting of respiratory syncytial virus F protein, cytomegalovirus, snake venoms and digoxin.

The term “binds to” as used herein, is intended to refer to the binding of an antigen by an immunoglobulin variable region of an antibody with a dissociation constant (Kd) of 1 μM or lower as measured by surface plasmon resonance analysis using, for example a BIAcore™ surface plasmon resonance system and BIAcore™ kinetic evaluation software (eg. version 2.1). The affinity or dissociation constant (Kd) for a specific binding interaction is preferably about 500 nM to about 50 pM, more preferably about 500 nM or lower, more preferably about 300 nM or lower and preferably at least about 300 nM to about 50 pM, about 200 nM to about 50 pM, and more preferably at least about 100 nM to about 50 pM, about 75 nM to about 50 pM, about 10 nM to about 50 pM.

The antibodies of the present invention are advantageous in human therapy because the likelihood of induction of a human anti-antibody response will be reduced.

Recombinant antibodies produced according tot he invention that bind a target antigen can be identified and isolated by screening a combinatorial immunoglobulin library (e.g., a phage display library) to isolate library members that exhibit the desired binding specificity and functional behaviour (for example neutralisation of TNFα can be measured using L929 cells). it will be understood that all approaches where antigen-binding portions or derivatives of antibodies are used, eg Fabs, scFv and V domains or domain antibodies, lie within the scope of the present invention. The phage display technique has been described extensively in the art and examples of methods and compounds for generating and screening such libraries and affinity maturing the products of them can be found in, for example, Barbas et al, 1991, Proc. Natl. Acad. Sci. USA, 88:7978-7982; Clarkson et al., 1991, Nature, 352:624:628; Dower et al., PCT Publication no. WO 91/17271, U.S. Pat. No. 5,427,908, U.S. Pat. No. 5,580,717 and EP 527,839; Fuchs et al., 1991, Bio/Technology, 9:1370-1372; Garrad et al., 1991 Bio/Technology, 9:1373:1377; Garrard et al., PCT Publication no. WO 92/09690; Gram et al., 1992, Proc. Natl. Acad. Sci. USA, 89:3576-3580; Griffiths et al., 1993 EMBO J, 12:725:734; Griffiths et al., U.S. Pat. No. 5,885,793 and EP 589,877; Hawkins et al, 1992, J Mol Biol, 226:889-896; Hay et al., 1992, Hum Antibod Hybridomas, 3:81-85; Hoogenboom et al., 1991 Nuc Acid Res, 19:4133-4137; Huse et al., 1989, Science, 246:1275-1281; Knappik et al., 2000, J Mol Biol, 296:57-86; Knappik et al. PCT WO 97/08320; Ladner et al. U.S. Pat. No. 5,223,409, No. 5,403,484, No. 5,571,698, No. 5,837,500 and EP 436,597; McCafferty et al., 1990, Nature, 348:552-554; McCafferty et al., PCT Publication no. WO 92/01047, U.S. Pat. No. 5,969,108 and EP 589,877; Salfeld et al., PCT WO 97/29131, U.S. Provisional Application No. 60/126,603; and Winter et al. PCT WO 92/20791 and EP 368,684;

Recombinant libraries expressing the antibodies of the invention can be expressed on the surface of microorganisms eg. yeast or bacteria (see PCT publications WO99/36569 and 98/49286).

The Selected Lymphocyte Antibody method or SLAM as it is referred to in the state of the art, is another means of generating high affinity antibodies rapidly. Unlike phage display approaches all antibodies are fully divalent. In order to generate New World primate antibodies, New World primates are immunised with a human antigen eg. a TNFα polypeptide. Following immunisation cells are removed and selectively proliferated in individual micro wells. Supernatants are removed from wells and tested for both binding and function. Gene sequences can be recovered for subsequent manipulations eg. humanisation, Fab fragment, scFv or dAb generation. Thus another example is the derivation of the ligand of the invention by SLAM and its derivatives (Babcook, J. S. et al 1996, Proc. Natl. Acad. Sci, USA 93; 7843-7848, U.S. Pat. No. 5,627,052 and PCT publication WO92/02551). Adaptations of SLAM, such as the use of alternatives to testing supernatants such as panning, also lie within the scope of this invention.

In one expression system the recombinant peptide/protein library is displayed on ribosomes (for examples see Roberts, R W and Szostak, J. W. 1997. Proc. Natl. Acad. Sci. USA 94:12297-123202 and PCT Publication No. WO98/31700). Thus another example involves the generation and in vitro transcription of a DNA library (eg of antibodies and derivatives) preferably prepared from immunised cells, but not so limited), translation of the library such that the protein and “immunised” mRNAs stay on the ribosome, affinity selection (eg by binding to RSP), mRNA isolation, reverse translation and subsequent amplification (eg by polymerase chain reaction or related technology). Additional rounds of selection and amplification can be coupled as necessary to affinity maturation through introduction of somatic mutation in this system or by other methods of affinity maturation as known in the state of the art (R. A. Irving et al. Journal of Immunological Methods, 248, 31-45 (2001)).

Another example sees the application of emulsion compartmentalisation technology to the generation of the antibodies of the invention. In emulsion compartmentalisation, in vitro and optical sorting methods are combined with co-compartmentalisation of translated protein and its nucleotide coding sequence in aqueous phase within an oil droplet in an emulsion (see PCT publication no's WO99026711 and WO0040712). The main elements for the generation and selection of antibodies are essentially similar to the in vitro method of ribosome display.

The antibody or antigen-binding portion thereof according to the invention can be derivatised or linked to another functional molecule. For example, the antibody or antigen-binding portion can be functionally linked by chemical coupling, genetic fusion, noncovalent association or otherwise, to one or more other molecular entities, such as another antibody, a detectable agent, a cytotoxic agent, a pharmaceutical agent, and/or a protein or peptide that can mediate association of the antibody or antigen-binding portion thereof with another molecule (such as a streptavidin core region or a polyhistidine tag).

Cytotoxic agents commonly used to generate immunotoxins include radioactive isotopes such as ¹¹¹In or ⁹⁰Y, selenium, ribonucleases, binding domain—deleted truncated microbial toxins such as Pseudomonas exotoxin or Diphtheria toxin, tubulin inhibitors such as calicheamicin (ozagamicin), maytansinoids (including DM-1), auristatins, and taxoids, ribosome inactivating proteins such as ricin, ebulin I, saporin and gelonin, and prodrugs such as melphalan.

Useful detectable agents with which an antibody or antigen-binding portion thereof may be derivatised include fluorescent compounds. Exemplary fluorescent detectable agents include fluorescein, fluorescein, isothiocyanate, rhodamine, 5-dimethylamine-1-napthalenesulfonyl chloride, phycoerythrin and the like. An antibody may also be derivatised with detectable enzymes such as alkaline phosphatase, horseradish peroxidase, glucose oxidase and the like. When an antibody is derivatized with a detectable enzyme, it is detected by adding additional reagents that the enzyme uses to produce a detectable reaction product. An antibody may also be derivatised with biotin, and detected through indirect measurement of avidin or streptavidin binding.

The present invention also extends to PEGylated antibodies or antibody-binding portion which provide increased half-life and resistance to degradation without a loss in activity (e.g., reduction in binding affinity) relative to non-PEGylated antibody polypeptides.

The antibody or antigen-binding portion as described herein can be coupled, using methods known in the art, to polymer molecules (preferably PEG) useful for achieving the increased half-life and degradation resistance properties. Polymer moieties which can be utilised in the invention can be synthetic or naturally occurring and include, but are not limited to, straight or branched chain polyalkylene, polyalkenylene or polyoxyalkylene polymers, or a branched or unbranched polysaccharide such as a homo- or heteropolysaccharide. Preferred examples of synthetic polymers which can be used in the invention include straight or branched chain poly(ethylene glycol) (PEG), poly(propylene glycol, or poly(vinyl alcohol) and derivatives or substituted forms thereof. Particularly preferred substituted polymers for linkage to antibodies as described herein include substituted PEG, including methoxy(polyethylene glycol). Naturally occurring polymer moieties which can be used in addition to or in place of PEG include lactose, amylose, dextran, or glycogen, as well as derivatives thereof which would be recognised by persons skilled in the art.

Derivatized forms of polymer molecules include, for example, derivatives which have additional moieties or reactive groups present therein to permit interaction with amino acid residues of the antibody polypeptides described herein. Such derivatives include N-hydroxylsuccinimide (NHS) active esters, succinimidyl propionate polymers, and sulfydryl-selective reactive agents such as maleimide, vinyl sulfone, and thiol. Particularly preferred derivatized polymers include, but are not limited to PEG polymers having the formulae: PEG-O—CH₂CH₂CH₂—CO₂—NHS; PEG-O—CH₂—NHS; PEG-O—CH₂CH₂—CO₂-NHS; PEG-S—CH₂CH₂—CO-NHS; PEG-O₂CNH—CH(R)—CO₂-NHS; PEG-NHCO—CH₂CH₂—CO—NHS; and PEG-O—CH₂—CO₂—NHS; where R is (CH₂)₄)NHCO₂(mPEG). PEG polymers can be linear molecules, or can be branched wherein multiple PEG moieties are present in a single polymer.

The reactive group (e.g., MAL, NHS, SPA; VS, or Thiol) may be attached directly to the PEG polymer or may be attached to PEG via a linker molecule.

The size of polymers useful in the invention can be in the range of between 500 Da to 60 kDa, for example, between 100 Da and 60 kDa, 10 kDa and 60 kDa, 20 kDa and 60 kDa, 30 kDa and 60 kDa, 40 kDa and 60 kDa, and up to between 50 kDa and 60 kDa. The polymers used in the invention, particularly PEG, can be straight chain polymers or may possess a branched conformation.

The polymer (PEG) molecules useful in the invention can be attached to an antibody or antigen-binding portion thereof using methods which are well known in the art. The first step in the attachment of PEG or other polymer moieties to an antibody polypeptide monomer or multimer of the invention is the substitution of the hydroxyl end-groups of the PEG polymer by electrophile-containing functional groups. Particularly, PEG polymers are attached to either cysteine or lysine residues present in the antibody polypeptide monomers or multimers. The cysteine and lysine residues can be naturally occurring, or can be engineered into the antibody polypeptide molecule. For example, cysteine residues can be recombinantly engineered at the C-terminus of an antibody polypeptide, or residues at specific solvent accessible locations in an antibody polypeptide can be substituted with cysteine or lysine.

The antibody may be linked to one or more molecules which can increase its half-life in vivo. These molecules are linked to the antibody at a site on the antibody other than the antigen building site, so that they do not interfere/sterically hinder the antigen-binding site. Typically, such molecules are polypeptides which occur naturally in vivo and which resist degradation or removal by endogenous mechanisms. It will be obvious to one skilled in the art that fragments or derivatives of such naturally occurring molecules may be used, and that some may not be polypeptides. Molecules which increase half life may be selected from the following:

(a) proteins from the extracellular matrix, eg. collagen, laminin, integrin and fibronectin;

(b) proteins found in blood, eg. fibrin α-2 macroglobulin, serum albumin, fibrinogen A, fibrinogen B, serum amyloid protein A, heptaglobin, protein, ubiquitin, uteroglobulin, β-2-microglobulin, plasminogen, lysozyme, cystatin C, alpha-1-antitrypsin and pancreatic kypsin inhibitor;

(c) immune serum proteins, eg. IgE, IgG, IgM;

(d) transport proteins, eg. retinol binding protein, α-1 microglobulin;

(e) defensins, eg. beta-defensin 1, Neutrophil defensins 1, 2 and 3;

(f)proteins found at the blood brain barrier or in neural tissues, eg. melanocortin receptor, myelin, ascorbate transporters;

(g) transferrin receptor specific ligand-Neuro pharmaceutical agent fusion proteins (see U.S. Pat. No. 5,977,307); brain capillary endothelial cell receptor, transferrin, transferrin receptor, insulin, insulin-like growth factor 1 (IGF 1) receptor, insulin-like growth factor 2 (IGF 2) receptor, insulin receptor;

(h) proteins localised to the kidney, eg. polycystin, type IV collagen, organic anion transporter K1, Heymarm's antigen;

(i) proteins localised to the liver, eg. alcohol dehydrogenase, G250;

(j) blood coagulation factor X;

(k) α-1 antitrypsin;

(l) HNF 1α;

(m) proteins localised to the lung, eg. secretory component (binds IgA);

(n) proteins localised to the Heart, eg. HSP 27;

(o) proteins localised to the skin, e.g. keratin;

(p) bone specific proteins, such as bone morphogenic proteins (BMPs) eg. BMP-2, -4, -5, -6, -7 (also referred to as osteogenic protein (OP-1 and -8 (OP-2);

(q) tumour specific proteins, eg. human trophoblast antigen, herceptin receptor, oestrogen receptor, cathepsins eg cathepsin B (found in liver and spleen);

(r) disease-specific proteins, eg. antigens expressed only on activated T-cells: including LAG-3 (lymphocyte activation gene); oseoprotegerin ligand (OPGL) see Nature 402, 304-309, 1999; OX40 (a member of the TNF receptor family, expressed on activated T cells and the only costimulatory T cell molecule known to be specifically up-regulated in human T cell luekaemia virus type-I (HTLV-I)-producing cells—see J. Immunol. 2000 July 1:16561):263-70; metalloproteases (associated with arthritis/cancers), including CG6512 Drosophila, human paraplegin, human FtsH, human AFG3L2, murine ftsH; angiogenic growth factors, including acidic fibroblast growth factor (FGF-1), basic fibroblast growth factor (FGF-2), Vascular endothelial growth factor/vascular permeability factor (VEGF/VPF), transforming growth factor-α(TGF-α), tumor necrosis factor-alpha (TNF-α), angiogenin, interleukin-3 (IL-3), interleukin-8 (IL-8), platelet derived endothelial growth factor (PD-ECGF), placental growth factor (PlGF), midkine platelet-derived growth factor-BB (PDGF), fractalkine;

(s) stress proteins (heat shock proteins);

(t) proteins involved in Fc transport; and

(u) vitamins eg B12, Biotin.

In another aspect, the invention provides a pharmaceutical composition comprising an effective amount of the antibody or antigen-binding portion thereof according to the present invention, together with a one or more pharmaceutically acceptable excipient or diluent.

A “pharmaceutically acceptable excipient or diluent” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol, and the like as well as combinations thereof. In many cases it will be preferable to include isotonic agents, for example, sugars, polyalcohols such as mannitol, sorbitol, or sodium chloride in the composition.

The term “effective amount” refers to an amount of an antibody or antigen binding portion thereof (including pharmaceutical compositions comprising the antibody or antigen binding portion thereof) sufficient to treat or ameliorate a specified disease or disorder or one or more of its symptoms and/or to prevent or reduce the occurrence of the disease or disorder.

The term “diagnostically effective amount” or “amounts effective for diagnosis” and cognates thereof, refers to an amount of a antibody or antigen binding portion thereof (including pharmaceutical compositions comprising the antibody or antigen binding portion thereof) sufficient to diagnose a specified disease or disorder and/or one or more of its manifestations, where diagnosis includes identification of the existence of the disease or disorder and/or detection of the extent or severity of the disease or disorder. Often, diagnosis will be carried out with reference to a baseline or background detection level observed for individuals without the disease or disorder. Levels of detection above background or baseline levels (elevated levels of detection) are indicative of the presence and, in some cases, the severity of the condition.

When used with respect to methods of treatment and the use of the antibody or antigen binding portion thereof (including pharmaceutical compositions comprising the antibody or antigen binding portion thereof), an individual “in need thereof” may be an individual who has been diagnosed with or previously treated for the disease or disorder to be treated. With respect to methods of diagnosis, an individual “in need thereof” may be an individual who is suspected to have a disease or disorder, is at risk for a disease or disorder, or has previously been diagnosed with the disease or disorder (e.g., diagnosis can include monitoring of the severity (e.g., progression/regression) of the disease or disorder over time and/or in conjunction with therapy).

It is preferred that the antibody or antigen-binding portion thereof blocks or stimulates receptors functions or neutralizes active soluble products, such as one or more of the interleukins, TNF or C5a. More preferably, the active soluble product is human TNF-α.

The composition may be in a variety of forms, including liquid, semi-solid or solid dosage forms, such as liquid solutions (eg injectable and infusible solutions), dispersions or suspensions, tablets, pills, powders, liposomes or suppositories. Preferably, the composition is in the form of an injectable solution for immunization. The administration may be intravenous, subcutaneous, intraperitoneal, intramuscular, transdermal, intrathecal, and intra-arterial. Preferably the dosage form is in the range of from about 0.001 mg to about 10 mg/kg body weight administered daily, weekly, bi- or tri-weekly or monthly, more preferably about 0.05 to about 5 mg/kg body weight weekly.

The composition may also be formulated as a sterile powder for the preparation of sterile injectable solutions.

In certain embodiments, the active compound may be prepared with a carrier that will protect the compound against rapid release, such as a controlled release formulation, including implants, transdermal patches, and microencapsulated delivery systems. Compatible polymers may be used such as ethylene vinyl acetate, polyanhydrides, polyglycolic acid, collagen, polyorthoesters or polylactic acid.

The composition may also be formulated for oral administration. In this embodiment, the antibody may be enclosed in a hard or soft shell gelatin capsule, compressed into tablets, or incorporated directly into the subject's diet.

The composition may also be formulated for rectal administration.

The antibody may be administered in order to bind to and identify selected cells in vitro and in vivo, to bind to and destroy selected cells in vivo, or in order to penetrate into and destroy selected cells in vivo. Alternatively, the antibody may be used as an immunotoxin to deliver a cytotoxic agent eg. a toxin or chemotherapeutic agent to a particular cell type such as a tumour cell. Production of immunotoxins would be familiar to persons skilled in the art.

In the preferred embodiment, the composition is administered to a human.

The present invention also provides for the use of the antibody or antigen-binding portion thereof in a diagnostic application for detecting an antigen associated with a particular disease or disorder.

More particularly, the invention provides for the use of the antibody or antigen-binding portion thereof in a method for diagnosing a subject having an antigen associated with a particular disease or disorder, comprising administering to said subject a diagnostically effective amount of an antibody, an antigen-binding portion thereof or pharmaceutical composition, as described herein, according to third aspect. Preferably the subject is a human.

The antibody or antigen-binding fragment thereof, preferably labelled, can be used to detect the presence of an antigen, or elevated levels of an antigen (e.g. TNF-α) in a biological sample, such as serum or plasma using a convention immunoassay, such as an enzyme linked immunosorbent assay (ELISA), a radioimmunoassay (RIA) or tissue immunohistochemistry.

Preferably, the antigen to which the chimeric antibody or antigen-binding portion thereof binds, is peptide, protein, carbohydrate, glycoprotein, lipid or glycolipid in nature, selected from a tumour-associated antigen including carcinoembryonic antigen, EpCAM, Lewis-Y, Lewis-Y/b, PMSA, CD20, CD30, CD33, CD38, CD52, CD154, EGF-R, Her-2, TRAIL and VEGF receptors, an antigen involved in an immune or inflammatory disease or disorder including CD3, CD4, CD25, CD40, CD49d, MHC class I, MHC class II, GM-CSF, interferon-γ, IL-1, IL-12, IL-13, IL-23, TNF-α, and IgE, an antigen expressed on a host cell including glycoprotein IIb/IIIa, P-glycoprotein, purinergic receptors and adhesion receptors including CD11a, CD11b, CD11c, CD18, CD56, CD58, CD62 or CD144, an antigen comprising a cytokine, chemokine, growth factor or other soluble physiological modulator or a receptor thereof including eotaxin, IL-6, IL-8, TGF-β, C3a, C5a, VEGF, NGF and their receptors, an antigen involved in central nervous system diseases or disorders including β-amyloid and prions, an antigen of non-human origin such as microbial, nanobial or viral antigens or toxins including respiratory syncitial virus protein F, anthrax toxin, rattle snake venom and digoxin; wherein the chimeric antibody acts as an agonist or antagonist or is active to either deplete (kill or eliminate) undesired cells (eg. anti-CD4) by acting with complement, or killer cells (eg. NK cells) or is active as a cytotoxic agent or to cause Fc-receptor binding by a phagocyte or neutralizes biological activity of its target.

The anti-human TNF-α antibody or antigen binding portion thereof according to the invention may also be used in cell culture applications where it is desired to inhibit TNF-α activity.

The present invention also provides a method for treating a disease or disorder characterised by human TNF-α activity in a human subject, comprising administering to the subject in need thereof an antibody, an antigen-binding portion thereof or a pharmaceutical composition, as described herein, according to the present invention in which the antibody or antigen-binding portion thereof binds TNF-α.

The term “disease or disorder characterised by human TNF-α activity” as used herein is intended to include diseases or disorders in which the presence of TNFα in a subject suffering from the disease or disorder has been shown to be or is suspected of being either responsible for or involved in the pathophysiology of the disease or disorder or a factor that contributes to the worsening of the disease or disorder. Accordingly, a disease or disorder in which TNF-α activity is detrimental is a disease or disorder in which inhibition of TNF-α activity is expected to alleviate symptoms and/or progression of the disease or disorder. Such disease or disorders may be evidenced, for example, by an increase in the concentration of TNF-α in a biological fluid of a subject suffering from the disease or disorder (e.g., an increase in the concentration of TNF-α in serum, plasma, synovial fluid etc of the subject), which can be detected, for example, using an antibody of the invention specific for TNF-α.

A disease or disorder characterised by human TNF-α activity is intended to include diseases or disorders in which the presence of TNF-α in a subject suffering from the disease or disorder has been shown to be, or is suspected of being, either responsible for the pathophysiology of the disease or disorder or a factor which contributes to a worsening of the disease or disorder. preferably, the disease or disorder characterised by human TNF-α activity is selected from the group consisting of sepsis, including septic shock, endotoxic shock, gram negative sepsis and toxic shock syndrome; autoimmune disease, including rheumatoid arthritis, rheumatoid spondylitis, osteoarthritis, psoriasis and gouty arthritis, allergy, multiple sclerosis, autoimmune diabetes, autoimmune uveitis and nephrotic syndrome; infectious disease, including fever and myalgias due to infection and cachexia secondary to infection; graft versus host disease; tumour growth or motastasis; pulmonary diseases including adult respiratory distress syndrome, shock lung, chronic pulmonary inflammatory disease, pulmonary sarcoidosis, pulmonary fibrosis and silicosis; inflammatory bowel diseases including Crohn's disease and ulcerative colitis; cardiac diseases; inflammatory bone diseases, hepatitis, coagulation disturbances, burns, reperfusion injury, keloid formation and scar tissue formation.

Supplementary active compounds can also be incorporated into the composition. The antibody or antibody-binding fragment may be co-formulated with and/or administered simultaneously, separately or sequentially with one or more additional therapeutic agents eg. antibodies that bind to other targets such as cytokines or ell surface molecules or alternatively one or more chemical agents that inhibit human TNF-α production or activity.

In another aspect, the invention provides a kit comprising a therapeutically effective amount of an antibody or antigen-binding portion of the invention, or a pharmaceutical composition comprising a therapeutically effective amount of an antibody or antigen-binding portion thereof, together with packaging and instructions for use. In certain embodiments, the instructions for use include instructions for how to effectively administer a therapeutic amount of an antibody or antigen-binding portion of the invention.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

All publications mentioned in this specification are herein incorporated by reference. Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia or elsewhere before the priority date of each claim of this application.

In order that the nature of the present invention may be more clearly understood, preferred forms thereof will now be described with reference to the following non-limiting examples.

### EXAMPLE 1

### Fusion of a Marmoset Variable Region to a Human Constant Region

Materials and Methods

Gene Synthesis and Cloning

The V_{H} chain (Accession Number: AAM54057, SEQ ID NO: 1) of the MOG specific marmoset derived antibody was expressed with a human constant region (human IgG1 heavy chain C_{H}1, hinge C_{H}2 & C_{H}3 domains (such as NCBI accession number P01857) (SEQ ID NO: 2)). This was achieved by back translation of the amino acid sequence into a DNA sequence which was optimized for mammalian cell expression using GeneOptimizer technology and synthesized de nova by assembly of synthetic oligonucleotides (Gene Art, Germany). During DNA sequence optimisation the specific restriction enzyme sites Asc I and Tth 111I were included to allow for future manipulation of the V_{H} region. Following gene synthesis the whole sequence including a Kozak sequence was cloned into the multiple cloning site of the pEE6.4 GS accessory vector (Lonza Biologics). The V_{L} chain (Accession Number: AAM54058, SEQ ID NO:3) of the MOG specific marmoset derived antibody was expressed with a human kappa light chain constant region (such as NCBI accession number AAA58989) (SEQ ID NO: 4). DNA encoding the light chain (V_{L}-Kappa) amino acid sequence was prepared as described above for the heavy chain. During DNA sequence optimization and synthesis the specific restriction enzyme sites Bsi WI/Rsr II were included to allow future manipulation of the V_{L} region. Following gene synthesis the whole sequence including a Kozak sequence was cloned into the multiple cloning site of the pEE12.4 GS expression vector (Lonza Biologics). For stable expression the two single gene vectors (pEE6.4-V_{H}-IgG₁ and pEE12.4-V_{L}-Kappa) were combined into a double gene vector. This was done by digesting out of the pEE6.4 backbone the heavy chain expression cassette (hCMV-MIE promoter, Kozak sequence, marmoset V_{H}, human constant region and SV40 polyA site) using Not I and BamH I. The resultant fragment was subcloned using Not I and BamH I sites into the pEE12.4-V_{L}-Kappa vector downstream of the light chain expression cassette (hCMV-MIE promoter, Kozak sequence, marmoset V_{L}, human Kappa constant region and SV40 polyA site) creating a vector expressing both the heavy and light chains of AB138 (SEQ ID NOs: 5 and 6).

Transfection

For each transfection 175 μl of Lipofectamine 2000 was added to 5 mL of Optimem I media (Invitrogen Cat Nos. 11668-027 and 31985-062) in a well of a 6 well plate. In a second well 70 μl of the expression vector (70 μg) was added to 5 mL of Optimem I media. Following a 5 minute room temperature incubation, the contents of the two wells were mixed together and left for a further 20 minute incubation. Following this second incubation the whole transfection mixture was added to a T175 tissue culture flask containing the CHOK1SV cells. Cells were incubated for 72 to 96 hours and supernatants harvested. Supernatants were centrifuged at 4,000×g for 5 minutes to pellet cell debris, and were filter sterilised through 0.22 μm cartridge filter.

Antibody Purification

The supernatant was passed over a IliTrap Protein A column (Amersham Biosciences, Cat No: 17-0402-01) three times at a flow rate of 1 mL/min. The column was then washed with 20 mM sodium phosphate for 50 mins at 1 mL/min. The antibody was eluted with 0.1 M citric acid pH 3.5 with fractions collected and immediately neutralised with 1 M Tris-HCl pH 9.0. Antibody samples were then desalted on a PD-10 column (Amersham Biosciences, Cat No: 17-0851-01). Analysis of the antibody by SDS-PAGE and size-exclusion HPLC confirmed the correct molecular weight, presence of assembled antibody and the concentration of antibody.

Western Blot Analysis

The ability of AB138 to retain binding to the antigen of M26, rat MOG (myelin-oligodendrocyte glycoprotein), was investigated by Western Blot. 130 mg of rat spinal cord (IMVS, Australia) was homogenized in 1.8 ml CelLytic M Cell Lysis Reagent (SIGMA, C2978) and incubated for 30 minutes at 4° C. Further homogenization was performed by drawing the lysate through a 27 g ½ needle several times followed by centrifugation at 4° C. and 13000 g for 30 minutes. The pellet and supernatant was diluted into SDS-PAGE sample buffer (125 mM Tris-HCl pH 6.8, 5% SDS, 0.25% bromophenol blue, 25% glycerol). Along with this 200 μl CHOK1SV cells at 1×10⁶ viable cells per ml were spun down at 13000×g at 4° C. for 1 minute and resuspended in 200 μl CelLytic M Cell Lysis Reagent (SIGMA). Following centrifugation at 4° C. and 13000×g for 30 minutes the supernatant was mixed with the appropriate amount of SDS-PAGE sample buffer. All samples, along with a sample of molecular weight markers, were run on a 4-20% Novex pre-cast gel (Invitrogen, Australia) for 2 hours at 120V. Proteins were then transferred to PVDF (BioRad, Australia) using a western blot apparatus in 1×Tris-Glycine Buffer with 20% methanol (BioRad, Cat 161+-0771) at 4° C. at 250 mA for 2 hours. The membrane was then blocked by incubation with 5% skim milk, powder in PBS for 1 h at room temperature. The membrane was then washed with 1×PBS three times, followed by an overnight incubation at 4° C. with AB138 in PBS at 10 ug/mL. After washing, the membrane was incubated with Goat Anti-human IgG (H+L) HRP conjugate (Sigma, Australia) diluted 1:5000 in 1×PBS for 1 hour at room temperature. Following washing, bound antibody was detected using the ECL Western Blotting Analysis System, (Amersham Biosciences Cat: RPN2109). A parallel experiment was performed in which AB138 was replaced with an isotype-matched irrelevant specificity negative control antibody (anti-TNFα monoclonal antibody) in order to identify any non-specific binding events.

Results

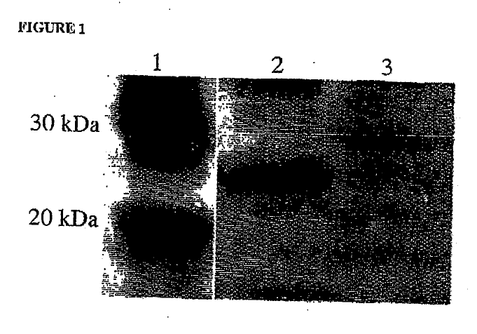

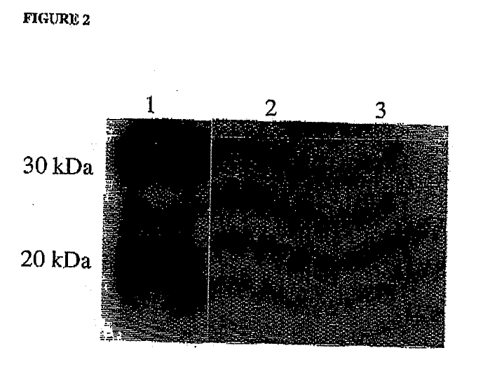

After successful protein expression and purification, western blot analysis was performed on AB138 to determine if it retained binding affinity to rat MOG. AB138 bound a protein with approximate size of 25 kDa present in the rat spinal cord cleared lysate, a protein not present in cleared CHOK1SV lysate (FIG. 1). The negative control antibody did not bind to protein present in either lysate indicating the interaction between AB138 and the protein of size 25 kDa was not due to artifact or non-specific binding events associated with the human constant region (FIG. 2). This protein matches the expected size of rat MOG minus the signal sequence (24.9 kDa). This result indicates that AB138 retained affinity for rat MOG present in rat spinal cord lysate and demonstrates that a marmoset human fusion antibody can retain antigen binding ability.

It can be appreciated by someone skilled in the art that rat MOG could be produced using recombinant DNA technology and the ability of AB138 to bind rat MOG determined in binding assays such as ELISA or Biacore analysis.

### EXAMPLE 2

### Engineering of a Monoclonal Antibody

1. Terminology

A donor sequence is defined as any immunoglobulin sequence derived from a species other than a New World primate.

An acceptor sequence is defined as an immunoglobulin sequence derived from a New World primate.

A common residue is a residue that is common (e.g. >30%) at a given amino acid position when determined by comparison with immunoglobulin sequences available for a species.

An uncommon residue is a residue that is uncommon (e.g. ≦30%) at a given amino acid position when determined by comparison with the immunoglobulin sequences available for a species.

Engineering is the process of transferring structural binding features of a donor sequence into an acceptor sequence such that the structural binding features maintain their binding activity.

A framework amino acid is defined as an amino acid located in an antibody variable region but not located in a CDR.

2. Abbreviations

CDR complementarity determining region, MOG, myelin/oligodendrocyte glycoprotein TNF-α, tumour necrosis factor—alpha; V_{H}, variable heavy chain; V_{L}, variable light chain; BSA, bovine serum albumin.

3. Engineering Process

A. Production of a monoclonal antibody (other than a New World primate monoclonal antibody).
B. Selection of an acceptor immunoglobulin sequence derived from a New World primate, on the basis of high amino acid sequence homology and predicted low immunogenicity.
C. Identification of the CDRs for both the donor and acceptor immunoglobulin sequences according to the numbering system of Kabat (See “Sequences of Proteins of Immunological Interest” E. Kabat et al., U.S. Department of Health and Human Services, 1983).
D. Determination of differences in the framework sequence by alignment of donor and acceptor sequences.
E. Prediction of donor immunoglobulin structure by three dimensional modelling and determination of proximity of the framework sequence differences relative to the CDRs. Optional substitution of acceptor residues with donor residues according to substitution criteria 1 & 2 (below)
F. Substitution of the entire acceptor CDR sequences with entire donor CDR sequences.
G. Determination of common residues by comparison of the donor/acceptor framework amino acid sequence with the germline and available acceptor immunoglobulin framework sequences. Optional substitution of acceptor residues with donor residues according to substitution criterion 3 & 4 (below)
H. Production of a chimeric antibody with acceptor variable regions and human constant regions
I. Expression of engineered immunoglobulin protein
J. Assay analysis of engineered immunoglobulin protein
   Substitution Criteria:

In generating a engineered antibody based on differences in the framework sequences, substitutions of an acceptor amino acid with the corresponding donor amino acid may be made at positions that fall into the following criteria:

(i) if the donor residue is predicted capable of interacting with the antigen based on three dimensional modelling;

(ii) if the donor residue is determined to live within 3.2 Å of the donor CDRs based on three dimension modelling;

(iii) if the donor residue is a common in acceptor species immunoglobulin sequences;

(iv) if the donor residue is uncommon in the donor germline.

The engineered antibody is predicted to be non-immunogenic or of low immunogenicity in humans by selecting appropriate acceptor sequences based on amino acid sequence homology with equivalent human sequences and predicted low immunogenicity. The engineered antibody will bind to the antigen of the donor immunoglobulin with a similar binding affinity to the donor immunoglobulin. The binding affinity of the engineered antibody can be further increased by methods of affinity maturation (R. A. Irving et al. Journal of Immunological Methods, 248, 31-45 (2001).

The Engineering of Murine Antibody AB164 to Yield Antibody AB197

4. Donor Immunoglobulin Sequences

Production of a murine hybridoma secreting a monoclonal antibody AB164 against human TNF-α was produced using hybridoma technology and served as the donor immunoglobulin sequences (SEQ ID NOs: 7 and 8).

5. Selection of Acceptor Immunoglobulin Sequences

The sequence of a monoclonal antibody against rat MOG (myelin/oligodendrocyte glycoprotein) was obtained from PubMed (http://www.ncbi.nlm.nih.gov/) and was used as the acceptor sequence. This monoclonal antibody was derived from a common marmoset (white tuffed-ear marmoset (Callithrix jacchus), a New World primate. The framework regions of the V_{H} chain (Accession Number: AAM54057, SEQ ID NO: 1) and the V_{L} chain (Accession Number: AAM54058, SEQ ID No: 3) were examined for their predicted immunogenicity in humans by the MHC class II binding prediction program Propred (http://www.intech.res.in/raghava/propred) using a 1% threshold value analysis of all alleles. A BLAST analysis of the sequence, excluding CDRs, of the V_{H} chain (Accession Number: AAM54057, SEQ ID NO: 1) and the V_{L} chain (Accession Number: AAM54058, SEQ ID No: 3) of the MOG specific antibody identified the closest human homologue heavy chain sequence (Accession Number AAH19337.1; SEQ ID NO: 9) and the light chain sequence (Accession Number: BAC53922.1; SEQ ID NO: 10).

Notably, this prediction analysis indicates that the selected acceptor heavy chain variable framework region is likely to be less immunogenic than its human equivalent. The acceptor heavy chain variable region had one peptide in the framework, LRPEDTAVY, which is predicted to bind MHC class II encoded by alleles DRB1_{—}0101, DRB1_{—}0102, DRB1_{—}0309. Whereas the closest human homologue heavy chain had three peptides, in the framework, that were predicted to bind to MHC class II. This included the peptide WVRQAPGQGL which is predicted to bind MHC class II encoded by alleles DRB1_{—}0101, DRB1_{—}0102 and DRB1 _{—}0309; the peptide VYMELTS which is predicted to bind MHC class II encoded by alleles DRB1_{—}0401, DRB1_{—}0408, DRB1_{—}0421, DRB1_{—}0426, DRB1_{—}1101, DRB1_{—}1128, DRB1_{—}1305; and the peptide LRSEDTAVY, which is predicted to bind MHC class II encoded by alleles DRB1_{—}0401, DRB1_{—}0421, DRB1_{—}0426.

The MOG specific light chain variable framework region and closest human homologue were predicted to be non-immunogenic.

6. Identification of the CDRs in the Donor/Acceptable Variable Regions

Using the rules of Kabat (See “Sequences of Proteins of Immunological Interest” E. Kabat et al., U.S. Department of Health and Human Services, 1983) the CDRs were determined for V_{H} and V_{L} chains of AB164 (SEQ ID NOs: 7 and 8 respectively) and for the V_{H} and V_{L} chains of the marmoset MOG specific immunoglobulin (SEQ ID No: 1 and 3 respectively)

TABLE 1

| | SEQ ID | | | |
|---|---|---|---|---|
| Chain | NO: | CDR-1 | CDR-2 | CDR-3 |
| V_{II} | 1 | 26-35 | 50-66 | 99-107 |
| V_{H} | 7 | 26-35 | 50-66 | 99-108 |
| V_{L} | 3 | 24-38 | 54-60 | 93-101 |
| V_{L} | 8 | 24-34 | 50-56 | 89-97 |

7. Alignment of Donor and Acceptor Sequences
V_{H} Chain Alignment

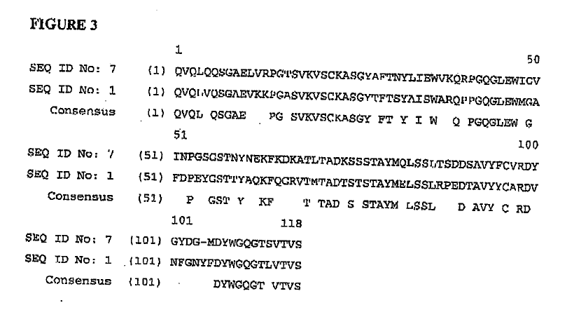

The amino acid sequences for the V_{H} chains of AB164 and MOG specific immunoglobulin (SEQ ID NOs: 7 and 1) were aligned (FIG. 3). The number of residues differs by one with an extra amino acid located in the CDR3 of the MOG specific immunoglobulin V_{H} chain. Sequence identity between the two sequences is 63.6%. The amino acid sequences of the CDRs differ as expected given the different antigen specificities of donor and acceptor antibodies. There are 22 amino acid differences between the sequences in the framework regions.

V_{L} Chain Alignment

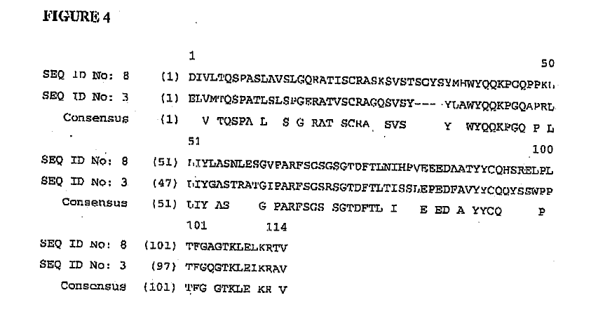

The amino acid for the V_{L} chains of AB164 and MOG specific immunoglobulin (SEQ ID No: 8 and 3) were aligned (FIG. 4). The number of residues differs by four additional amino acids located in the CDR1 of AB164. Sequence identity between the two sequences is 62.3%. The amino acid sequences of the CDRs differ as expected given the different antigen specificities of donor and acceptor antibodies. There are 23 amino acid differences between the sequences in the framework regions.

8. Predicted Three-Dimensional Modelling of the V_{H} and the V_{L} Chains of AB164

Using SWISS-PROT three-dimensional prediction modelling software and Deep View (http://swissmodel.expasy.org/) a three-dimensional model of the V_{H} and V_{L} chains of AB164 was determined. The CDRs were identified. The amino acid differences between the donor and acceptor sequences in the framework region, as determined by alignment described previously, were identified and a prediction made on their proximity to the CDRs (Tables 3 and 4)

9. Substitution of Acceptor CDRs with Donor CDRs

The CDRs of the V_{H} and V_{L} chains of MOG specific immunoglobulin were replaced with CDRs of the V_{H} and V_{L} chains of AB164 (Table 2)

TABLE 2

The replacement of the CDRs of the acceptor
sequence (MOG specific immunoglobulin) with
the CDRs of the donor sequence (AB164)

| | | Acceptor sequence | Replaced with |
|---|---|---|---|
| Chain | CDR | MOG specific IgG | AB164 sequence |
| V_{H} | 1 | GYTFTSYAIS | GYAFTNYLIE |
| V_{H} | 2 | AFDPEYGSTTYAQKFQG | VINPGSGSTNYNEKFKD |
| V_{H} | 3 | DVNFGNYFDY | DYGYDGMDY |
| V_{L} | 1 | RAGQSVSYYLA | RASKSVSTSGYSYMH |
| V_{L} | 2 | GASTRAT | LASNLES |
| V_{L} | 3 | QQYSSWPPT | QHSRELPLT |

10. Determining Common Residues in the Murine Germline and Marmoset Ig Sequences and Selection of Engineered Framework Sequence
V_{H} Chain

The murine germline alignment of V_{H} regions can be found at http://www.ibt.unam.mx/vir/vh_mice_directory.html#GL.

Marmoset V_{H} sequences can be obtained from http://www.ncbi.nlm.nih.gov/entrez/query.fcgi?db=Protein&itool=toolbar

by searching for all V_{H} amino acid sequences from Callithrix jacchus and aligning these sequences. Using alignment tools the common residues in both the murine germlines and the available Callithrix jacchus sequences were determined at each amino acid position where a difference in amino acids in the framework sequence between donor and acceptor sequence occurred (Table 3)

TABLE 3
V_{H} framework differences in the donor/acceptor sequence, their proximity to
the CRRs and their relative common residues in the donor/acceptor species. A
determination of the common residues at each position in the respective
murine germline and the available marmoset V_{H} sequences was performed.
At selected positions that satisfied a particular criteria the acceptor amino acid
was replaced with a donor amino acid and the number of that criteria is given;

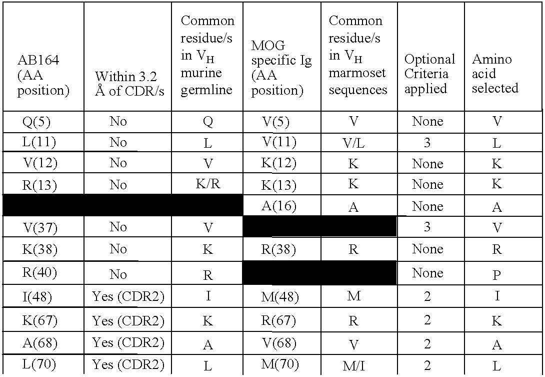

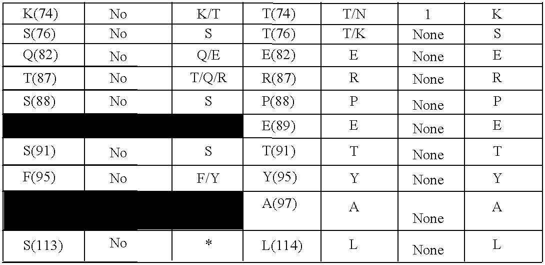

1. if the donor residue is predicted capable of interacting with the antigen based on three dimensional modelling;
2. if the donor residue is determined to lie within 3.2 Å of the donor CDRs based on three dimensional modelling;
3. if the donor residue is a common residue in acceptor species immunoglobulin sequences;
4. if the donor residue is uncommon in the donor germline.

At positions that fail the criteria the acceptor sequence was used and the criteria listed as None.

Note: Uncommon residues are in shaded in grey and substitutions are in bold. *Murine germline contains no sequence data at position 113 and as such marmoset sequence was used here.

In summary, there were 8 framework amino acid substitutions in which acceptor sequence was replaced with donor sequence. There were four amino acids in which the acceptor sequence was substituted with the donor sequence because the donor residue was determined to lie with 3.2 Å of the donor CDRs, based on three dimensional modelling. Two amino acid substitutions were made because the donor residues were predicted capable of interacting with the antigen being located on the turn of a loop that is in close proximity (though not less than 3.2 Å) with CDR-2. Further, two amino acid substitutions were made because the donor residue was found to be common in the acceptor species immunoglobulin sequences available. A further change could also be made at position 97.

V_{L} Chain

The murine germline alignment of V_{L} regions can be found at http://www.ibt.unam.mx/vir/vk_mice_directory.html#GLvk

Marmoset V_{L} sequences can be obtained from http:///www.ncbi.nlm.nih.gov/entrez/query.fcgl?db=Protein&itool=toolbar by searching for all amino acid sequences from Callithrix jacchus and aligning these sequences. Using alignment tools the common residues in the murine germline and the available marmoset immunoglobulin sequences were determined at each amino acid position relative to differences in amino acids in the framework sequence between donor and acceptor sequence (Table 4)

TABLE 4
V_{L} framework differences in the donor/acceptor sequence, their
proximity to the CRRs and their relative common residues in the donor/acceptor
species. A determination of the common residues at each position in the
respective murine germline and the available marmoset V_{L} sequences
respective murine position the criteria for selecting differences in
framework sequence given above was applied. At a position that satisfied a
particular criteris the acceptor amino acid was replaced with a donor amino
acid and the number of that criteria is given;

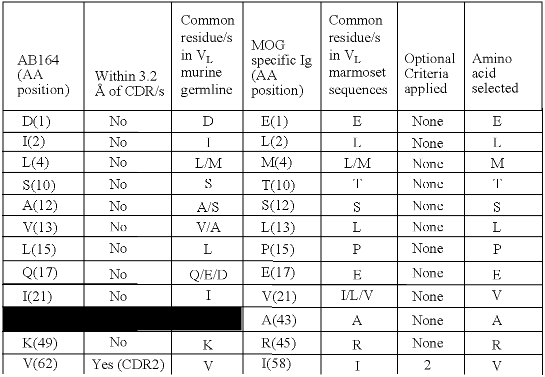

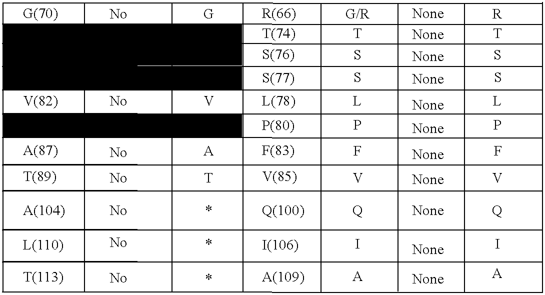

1. if the donor residue is predicted capable of interacting with the antigen based on three dimensional modelling;
2. if the donor residue is determined to lie within 3.2 Å of the donor CDRs based on three dimensional modelling;
3. if the donor residue is a common residue in acceptor species immunoglobulin sequences;
4. if the donor residue is uncommon in the donor germline.

At positions that fall the criteria the acceptor sequence was used and the criteria listed as None.

Note: Uncommon residues are in shaded in grey and substitutions are in bold. *Murine germline contains no sequence data at position 104 and beyond and as such marmoset sequence was used here.

In summary, there was 1 framework amino acid substitution in which acceptor sequence was replaced with donor sequence as the donor residue was determined to lie within 3.2 Å of the donor CDRs based on three dimensional modelling.

Materials and Methods

The AB164 hybridoma was generated by fusion of splenocytes from mice immunized with human TNF-α, with the myeloma cell line SP2/0-Ag14 by standard methods (Fazekas de St. Groth, S., et al. Journal of Immunological Methods 35: 1-21 (1980); Sugasawara, R., Journal of Tissue Culture Methods 12: 93-95 (1989)).

11. Sequence of Monoclonal Antibody AB164

Total RNA (tRNA) was extracted from 1×10⁷ to 1×10⁸ viable cells using RNeasy Mini or Midi columns (QIAgen) according to the manufacturer's instructions. Following quantitation, the tRNA was used as a template for first strand cDNA synthesis using an oligo(dT) primer and Superscript II Reverse Transcriptase (Invitrogen) according to manufacturer's instructions. Finally the tRNA was degraded using RNase H and the remaining single stranded cDNA tagged with a poly-G tail using terminal transferase and dGTP (Roche).

PCR reactions were performed using Herculase (Stratagene), a high fidelity polymerase blend. In each case an oligo (dC) was used as the forward primer with an IgG₁ heavy chain specific or a Kappa light chain specific reverse primer. Following 30 cycles PCR reactions were incubated in the presence of Taq polymerase to add overhanging A bases. The resulting PCR product was then cloned into pGemT-Easy (Promega) and transformed into competent Top 10 E. coli cells (Invitrogen). Plasmids were extracted from overnight culture of single colonies using QIAquick Miniprep columns (QIAgen) and quantified. 100 to 500 ng were mixed in duplicate with 6.4 pmol of either pUC3 forward or pUC3 reverse primer and submitted to cycle sequencing using BigDye v3.1 chemistry (AppliedBiosystems). Electrophoretograms were resolved on ABI PRISM 3700 DNA Analyser and following alignment of derived sequences, manual correction of aberrant base calling was performed. Once four matching sequences (2 forward and 2 reverse) were obtained the sequence of the antibodies variable region was confirmed. These sequences were then translated into amino acid sequences for the heavy and light chains of AB164 (SEQ ID NOS: 7 and 8)

12. Creation of AB138 (MOG Specific Marmoset Derived Variable Region—Human Constant Region Chimera) and AB103 (Anti-TNFα Murine Variable Region—Human Constant Region Chimera)

The V_{H} region (Accession Number: AAM54057, SEQ ID No: 1) of the acceptor sequence was expressed with a human constant region (human IgG1 heavy chain C_{H}1, hinge, C_{H}2 & C_{H}3 domains (such as NCBI accession number P01857) (SEQ ID No:2). The V_{L} region (Accession Number: AAM54058, SEQ ID No: 3) of the acceptor sequence as expressed with a human kappa light chain constant domain (such as NCBI accession number AAA58989) (SEQ ID No:4). The resultant chimeric antibody was designated AB138 (SEQ ID NOs: 5 and 6). This antibody was used as a template into which alterations in the V_{H} and V_{L} chains were made.

V_{H} and V_{L} regions from the fully murine AB164 (SEQ ID No: 7 and 8) were expressed with the same human constant regions as described above. This chimeric antibody was given the designation AB103.

Cloning of AB103

The V_{H} and V_{L} regions from the fully murine AB164 (SEQ ID No: 7 and 8) were back translated into DNA sequences which were optimized for mammalian cell expression using GeneOptimizer technology and synthesized de novo by assembly of synthetic oligonucleotides (GeneArt, Germany). For the V_{H} gene each sequence as flanked at the 5′ end with a Asc I site, a Kozak sequence (GCCACC) and a human IgG gamma leader sequence (amino acid sequence MEWSWVFLFFLSVTTGVHS). At the 3′ end the DNA sequence was manipulated to introduce a Tth 111I restriction enzyme site without compromising the required amino acid sequence. For the V_{L} gene each sequence as flanked at the 5′ end with a Bsi WI site, a Kozak sequence (GCCACC) and a human Kappa leader sequence (amino acid sequence MSVPTQVLGLLLLWLTDARC). At the 3′ end DNA sequence was manipulated to introduce a Rsr II restriction enzyme site without comprising the required amino acid sequence. Following de novo gene synthesis, the variable regions were provided cloned into a pCRScript vector (Stratagene) and were released by Asc I/Tth 111I and Bsi WI/Rsr II digestion for the V_{H} and V_{L} sequences respectively. Released sequences were ligated into single gene vector backbones derived from the vectors created to express AB138 prepared by Asc I/Tth 111I for pEE6.4-V_{H}-IgG₁ and Bsi WI/Rsr II for pEE12.4-v_{L}-Kappa digestion.

Each gene was ligated into the prepared backbone using the LigaFast Rapid DNA Ligation System from Promega (Cat No. M8221). Ligations were then transformed into One Shot Top 10 (chemically competent cells (Invitrogen Cat No. C4040-03) and positive colonies identified by standard techniques. A double gene vector for stable expression was prepared as outlined above (Example 1). Large quantities of the resulting vectors were prepared by midiprep of overnight cultures using QIAfilter midiprep columns (QIAgen Cat No. 12243). Vectors were prepared for transfection by precipitating 20 μg in 100% ethanol with 1/10 volume of 3M sodium acetate (pH5.2) (Sigma Cat Nos. E7023-500ML and S2889 respectively). Following a wash in 70% ethanol vectors were resuspended in 40 μl of T.E. pH8.0 (Sigma Cat No. T9285-100ML) at a working concentration of 0.5 μg/μl.

13. Creation of Engineered Monoclonal Antibody AB197

Using the MOG specific immunoglobulin as an acceptor sequence and by replacing the CDRs and nominated residues in the framework with those of the donor sequence (AB164), the engineered V_{H} and V_{L} antibody sequences were determined. These variable region protein sequences were expressed with human constant regions (SEQ ID NOs: 2 and 4). The resultant engineered antibody was designated AB197 (SEQ ID NOs: 11 and 12).

Table 5 describes the species origin of the CDRs, V_{H}/V_{L} framework and the constant regions for each antibody.

TABLE 5
Species origin of the CDRs, V_{II}/V_{L} framework and the
constant regions for AB138, AB164, AB197, AB103

| | | V_{H}/V_{L} | Constant | |
|---|---|---|---|---|
| Construct | CDRs | framework | regions | Antigen |
| AB138 | marmoset | marmoset | human | rat MOG |
| AB164 | murine | murine | murine | human TNFα |
| AB197 | murine | marmoset | human | human TNFα |
| AB103 | murine | murine | human | human TNFα |

Cloning of AB197

By replacing the CDRs and nominated residues in the framework of the acceptor sequence with those of the donor sequence, the engineered V_{H} and V_{L} antibody sequences were determined (SEQ ID No:11 and 12). The antibody sequence was back translated in DNA sequences and synthesized de novo by assembly of synthetic oligonucleotides (GeneArt, Germany). During synthesis of the relevant restriction enzyme sites were incorporated in the sequence to allow cloning and the generation of a double gene vector expressing AB197 as described previously (Example 1).

14. Expression of AB103, AB197 and AB164

Transfection of AB103 and AB197

For each transfection 175 μl of Lipofectamine 2000 was added to 5 mL of Optimem I media (Invitrogen Cat Nos. 11668-027 and 31985-062) in a well of a 6 well plate. In a second well 70 μl of the expression vector (70 μg) was added to 5 mL of Optimem I media. Following a 5 minute room temperature incubation, the contents of the two wells were mixed together and left for a further 20 minute incubation. Following this second incubation the whole transfection mixture was added a T175 tissue culture flask containing the CHOK1SV cells. Cells were incubated for 72 to 96 hours and supernatants harvested. Supernatants were centrifuged at 4,000×g for 5 minutes to pellet cell debris, and were filter sterilised through 0.22 μm cartridge filter.

Production of Murine Monoclonal Antibody AB164

Hybridoma cells expressing AB164 were cultured using standard tissue culture methods and the supernatant harvested and centrifuged at 4,000×g for 5 minutes to pellet cell debris followed by filter sterilisation through 0.22 μm cartridge filters.

Antibody Purification of AB103, AB197 and AB164

The supernatant was passed over a HiTrap Protein A column (Amersham Biosciences, Cat No: 17-0402-01) three times at a flow rate of 1 mL/min. The column was then washed with 20 mM sodium phosphate for 40 mins at 1 mL/min. The antibody was eluted with 0.1 M citric acid pH 3.5 with fractions collected and immediately neutralised with 1M Tris-HCl pH 9.0. Antibody samples were then desalted on a PD-10 column (Amersham Biosciences, Cat No: 17-0851-01). Analysis of the antibody by SDS-PAGE and size-exclusion HPLC confirmed the molecular weight, presence of assembled antibody and the concentration of antibody.

15. Affinity Binding Assays

Methods

ELISA Methods

TNF-α (Peprotech Cat No: 300-01A) was diluted to 1 μg/mL in carbonate coating buffer (10 mM disodium phosphate 20 mM sodium hydrogen phosphate pH 9.6). 100 μl of this solution was added to each well of a 96 well plate and incubated at 4° C. overnight in a humidified container. The plate was then washed three times with wash buffer (0.01M PBS pH 7.2, 0.05% Tween-20) and then three times with 0.01M PBS pH 7.2. The wells were then blocked by adding 200 μL blocking buffer (1% w/v BSA in 0.01M PBS pH 7.2) to each well and incubating the plate at 25° C., in a humidified container, for 1 hour. The antibody was diluted in antibody diluent (1% w/v BSA, 0.05% Tween-20 in 0.01M PBS pH 7.2) sufficient to generate a titration curve covering the ranges 6.00 μg/mL to 0.0578 ng/mL. The wells were incubated with the antibody for 1 hour at 25° C. The plate was then washed as previously described. 100 μL of Anti-IgG H+L antibody HRP conjugate (Zymed, Cat No: 81-71200) at 1:2000 in antibody diluent was used to detect bound AB197 and AB103. 100 μL of Anti-murine immunoglobulin antibody HRP conjugate (Dako, Cat No: P0260) at 1:2000 in antibody diluent was used to detect bound AB164. Wells with antibody diluent only were used to measure the background absorbance. After incubation at 25° C., in a humidified container, for 1 hour the plate was washed again as previously described. 100 μL TMB substrate solution (Zymed, Cat No: 00-2023) was added to each well and the colour allowed to develop for 4 min. 100 μL of 1M HCl was added to terminate the colour development reaction and absorbance was determined at 450 nm (ref. 620 nm)

ELISA Results

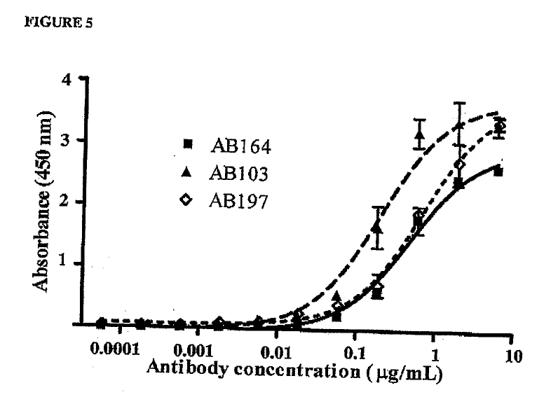

ELISA was used to compare the binding of AB164, AB197 and AB103 to TNF-α coated in the solid phase. From these results all antibodies displayed strong binding for TNF-α with all EC50 values less or equal to 0.68 μg/ml (FIG. 5, Table 6). The replacement of a murine constant region (AB164) with human IgG₁ constant (AB103) region did not significantly lower the binding affinity as can be seen by comparison of the binding profiles of the antibodies AB164 and AB103. Engineering of AB164 to yield AB197 did not result in any significant loss of TNF-α binding, as can be seen by comparison of the binding profiles of the antibodies AB164 and AB197. (FIG. 5)

TABLE 6

| Construct | EC-50 (μg/ml) |
|---|---|
| AB164 | 0.45 |
| AB197 | 0.68 |
| AB103 | 0.19 |

TNF-α Cytotoxicity Neutralisation Assay Using Live Cells (L-929 Neutralisation Assay) Method

L929 cells (ATCC No: CCL-1) were cultured in RPMI 1640 (Invitrogen Cat No: 21870-076) containing 10% foetal bovine serum, 50 μg/mL Penicillin/Streptomycin (Sigma Cat No: P0781), 2 mM L-glutamine (Invitrogen Cat No: 25030-081) and 10 μM 2-mercaptoethanol (Invitrogen Cat No: 21985-023) till the cells reached a 70% level of confluence. Into each well of a 96-well tissue culture plate 50 μL media was added.

To investigate the cytotoxicity of TNF-α on L929 cells, 50 μL of TNF-α working solution per well (30 ng/mL) was added to the first column of the plate in triplicate with serial half log dilutions performed across the plate reaching a final concentration of 9 fg/mL. Control wells with 50 μL media without TNF-α were also prepared (V=100%). To all wells 50 μL of L929 cells at 5×10⁻⁵ cells/mL was added. Further control wells were also prepared containing 100 μL of media with no additional cells or TNF-α (background). To all wells Actinomycin D (Sigma Cat No: A1410) at 40 μg/mL was added.

To investigate neutralisation by engineered antibodies against TNF-α a neutralisation assay was performed. 23 μL of antibody at 10 μg/mL was added to the first column of a separate plate in triplicate and serial log dilutions were performed across the plate reaching a final concentration of 30.4 pg/mL. To these wells 50 μL of L-929 cells at 5×10⁻⁵ cells/mL was added. A further 25 μL of Actinomycin-D was added to all wells.

All plates were incubated at 37° C. with 5% CO₂ for 20 hours. Following incubation 25 uL MTS/PES CellTiter 96 AQ_{ucous} One Solution Reagent (Promega Cat No: G358B) was added to all wells and incubated for 2 hours at 37° C. The absorbance was read at 492 nm (ref. 630 nm) using an ELISA plate reader. Average absorbance of all replicate treatments was subtracted from the average absorbance of the no cell and no TNF control wells (background). From this the % Viability of L-929 cells was calculated as:

$$\% Viability = \frac{{A}_{492} experimental wells}{{A}_{492} V = 100 \% viable} \times 100$$

TNFα Cytotoxicity Neutralisation Assay Using Live Cells (L-929 Neutralisation Assay) Results

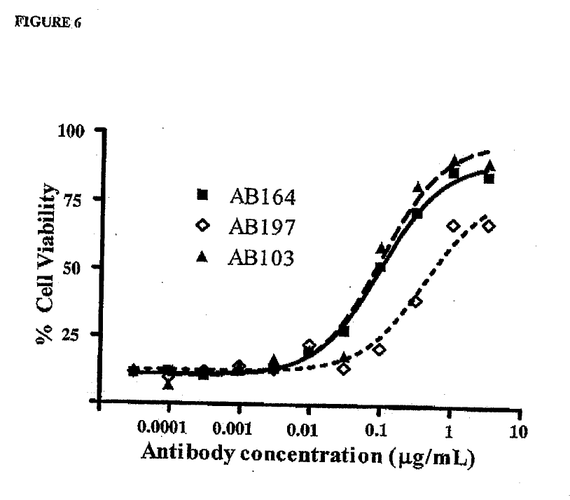

AB164, AB197 and AB103 were able to neutralise TNF-α-induced cytotoxicity (FIG. 6, Table 7)

TABLE 7

| Construct | EC-50 (μg/ml) |
|---|---|
| AB164 | 0.10 |
| AB197 | 0.41 |
| AB103 | 0.10 |

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1: An antibody or antigen-binding portion thereof having a variable region comprising at least two complementarity determining regions (CDRs) and at least three framework regions, wherein the framework regions are, or are derived from New World primate framework regions, and wherein at least one of the CDRs is a non-New World primate CDR.

2: An antibody or antigen-binding portion thereof according to claim 1 wherein the variable region comprises three CDRs and four framework regions.

3: An antibody or antigen-binding portion thereof according to claim 1 wherein the variable region comprises at least one murine CDR sequence.

4: An antibody or antigen-binding portion thereof according to claim 1 wherein the variable region comprises at least one mouse CDR sequence.

5: An antibody or antigen-binding portion thereof according to claim 1 wherein the variable region comprises at least one rat CDR sequence.

6: An antibody or antigen-binding portion thereof according to claim 1 wherein the variable region comprises at least one human CDR sequence.

7: An antibody or antigen-binding portion thereof according to claim 1 wherein the variable region comprises at least one synthetic CDR sequence.

8: An antibody or antigen-binding portion thereof according to claim 1 wherein the variable region comprises at least one rabbit CDR sequence.

9: An antibody or antigen-binding portion thereof according to claim 1 wherein the variable region comprises a combination of CDRs from differing sources.

10: An antibody or antigen-binding portion thereof according to claim 1 wherein the variable region comprises 3 murine CDR sequences.

11: An antibody or antigen-binding portion thereof according to claim 10 wherein the 3 murine CDR sequences are mouse CDR sequences.

12: An antibody or antigen-binding portion thereof according to claim 1 wherein the variable region comprises 3 human CDR sequences.

13: An antibody or antigen-binding portion thereof according to claim 1 wherein the variable region comprises 4 New World primate framework sequences.

14: An antibody or antigen-binding portion thereof according to claim 1 wherein the variable region comprises 4 framework regions in which the framework regions are derived from New World primate framework regions.

15: An antibody or an antigen-binding portion thereof according to claim 1 wherein the antigen-binding portion is a domain antibody.

16: An antibody or an antigen-binding portion thereof according to claim 1 wherein the antibody or antigen-binding portion further comprises a human or non-human Old World primate constant region sequence.

17: An antibody or antigen-binding portion thereof according to claim 1 wherein the New World primate framework regions are from a New World primate selected from the group consisting of marmosets, tamarins, squirrel monkey, titi monkey, spider monkey, woolly monkey, capuchin, uakaris, sakis, night or owl monkey and the howler monkey.

18: An antibody or antigen-binding portion thereof according to claim 17 wherein the New World primate is a marmoset.

19: An antibody or antigen-binding portion according to any claim 1 wherein the antibody or antigen-binding portion binds to an antigen that is peptide, protein, carbohydrate, glycoprotein, lipid or glycolipid in nature, selected from a tumour-associated antigen including carcinoembryonic antigen, EpCAM, Lewis-Y, Lewis-Y/b, PMSA, CD20, CD30, CD33, CD38, CD52, CD154, EGF-R, Her-2, TRAIL and VEGF receptors, an antigen involved in an immune or inflammatory disease or disorder including CD3, CD4, CD25, CD40, CD49d, MHC class I, MHC class II, GM-CSF, interferon-γ, IL-1, IL-12, IL-13, IL-23, TNF-α, and IgE, an antigen expressed on a host cell including glycoprotein IIb/IIIa, P-glycoprotein, purinergic receptors and adhesion receptors including CD11a, CD11b, CD11c, CD18, CD56, CD58, CD62 or CD144, an antigen comprising a cytokine, chemokine, growth factor or other soluble physiological modulator or a receptor thereof including eotaxin, IL-6, IL-8, TGF-β, C3a, C5a, VEGF, NGF and their receptors, an antigen involved in central nervous system diseases or disorders including β-amyloid and prions, an antigen of non-human origin such as microbial, nanobial or viral antigens or toxins including respiratory syncitial virus protein F, anthrax toxin, rattle snake venom and digoxin; wherein the chimeric antibody acts as an agonist or antagonist or is active to either deplete (kill or eliminate) undesired cells (eg. anti-CD4) by acting with complement, or killer cells (eg. NK cells) or is active as a cytotoxic agent or to cause Fc-receptor binding by a phagocyte or neutralizes biological activity of its target.

20: An antibody or antigen-binding portion thereof according to claim 19 wherein the antigen is human TNFα.

21: An antibody or antigen-binding portion thereof according to claim 1 wherein the sequence of at least one framework region is modified to increase binding.

22: An antibody or antigen-binding portion thereof according to claim 1 wherein the sequence of at least one framework region is modified to decrease predicted immunogenicity in humans.

23: A kit comprising an antibody or an antigen-binding portion thereof according to claim 1, or a pharmaceutical composition thereof, packaging and instructions for use.

24: A designed New World primate antibody or antigen-binding portion thereof which binds a cell surface antigen or a cytokine wherein the antibody or antigen-binding portion thereof comprises a variable region comprising at least two complementarity determining regions (CDRs) and at least three framework regions, wherein the CDRs are selected such that the antibody or antigen-binding portion binds to the cell surface antigen or to the cytokine.

25: A designed New World primate antibody or antigen-binding portion thereof as claimed in claim 24 wherein the antibody or antigen-binding portion thereof binds to a cell surface antigen selected from the group consisting of CD3, CD20, CD33, EGF-R, Her-2 and CD25.

26: A designed New World primate antibody or antigen-binding portion thereof as claimed in claim 24 wherein the antibody or antigen-binding portion thereof binds to TNFα or VEGF.

27: A designed New World antibody or an antigen-binding portion thereof according to claim 24 wherein the antigen-binding portion is a domain antibody.

28: A designed New World antibody or an antigen-binding portion thereof according to claim 24 wherein the antibody or antigen-binding portion further comprises a human or non-human Old World primate constant region sequence.

29: A designed New World antibody or antigen-binding portion thereof according to claim 24 wherein the New World primate is selected from the group consisting of marmosets, tamarins, squirrel monkey, titi monkey, spider monkey, woolly monkey, capuchin, uakaris, sakis, night or owl monkey and the howler monkey.

30: A designed New World antibody or antigen-binding portion thereof according to claim 29 wherein the New World primate is a marmoset.

31: A designed New World antibody or antigen-binding portion thereof according to claim 24 wherein the sequence of at least one framework region is modified to increase binding.

32: A designed New World antibody or antigen-binding portion thereof according to claim 24 wherein the sequence of at least one framework region is modified to decrease predicted immunogenicity in humans.

33: A kit comprising a designed New World antibody or an antigen-binding portion thereof according to claim 24, or a pharmaceutical composition thereof, packaging and instructions for use.
